SEQUENCE LISTING (11 sequences)

SEQ ID NO: 1 - protein, 417 aa, Homo sapiens
MPSSVSWGILLLAGLCCLVPVSLAEDPQGDAAQKTDTSHHDQDHPTFNKITPNLAEFAFSYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - protein, 38 aa, Homo sapiens
PMSIPPEVKFNKPFVFLMIEQNTKSPLFMGKVVNPTQK

SEQ ID NO: 3 - protein, 5 aa, Homo sapiens
PMSIP

SEQ ID NO: 4 - protein, 11 aa, Homo sapiens
SIPPEVKFNKP

SEQ ID NO: 5 - DNA, 29 bp, Artificial Sequence
  Description of Artificial Sequence Synthetic primer
gaggatccccagggagatgctgcccagaa

SEQ ID NO: 6 - DNA, 34 bp, Artificial Sequence
  Description of Artificial Sequence Synthetic primer
cgcgctcgagttatttttgggtgggattcaccac

SEQ ID NO: 7 - DNA, 30 bp, Artificial Sequence
  Description of Artificial Sequence Synthetic oligonucleotide
gcttgacctgtaactcgggccaggcgagct

SEQ ID NO: 8 - DNA, 30 bp, Artificial Sequence
  Description of Artificial Sequence Synthetic oligonucleotide
cgcctagcccgagttacaggtcaagcagct

SEQ ID NO: 9 - DNA, 42 bp, Artificial Sequence
  Description of Artificial Sequence Synthetic oligonucleotide
ctagaggatcccatggactacaaggacgacgatgacaaggaa

SEQ ID NO: 10 - DNA, 42 bp, Artificial Sequence
  Description of Artificial Sequence Synthetic oligonucleotide
gatcttccttgtcatcgtcgtccttgtagtccatgggatcct

SEQ ID NO: 11 - protein, 5 aa, Homo sapiens
FVFLM

(19) United States
(12) Patent Application Publication
(10) Pub. No.: US 2018/0092964 A1
(43) Pub. Date: Apr. 5, 2018

(54) LDL QUANTITATION AND METHODS OF USE

(71) Applicant: Cynthia L. Bristow, Long Beach, CA (US)

(72) Inventor: Cynthia L. Bristow, Long Beach, CA (US)

(21) Appl. No.: 15/844,353

(22) Filed: Dec. 15, 2017

Related U.S. Application Data
(63) Continuation of application No. 13/948,446, filed on Jul. 23, 2013, now abandoned.
Application No. 13/948,446 is a continuation of application No. 13/492,294, filed on Jun. 8, 2012, now abandoned.
Application No. 13/492,294 is a continuation of application No. PCT/US2010/059664, filed on Sep. 12, 2010.
(60) Provisional application No. 61/267,975, filed on Dec. 9, 2009.

(51) Int. Cl. A61K 38/17; A61K 45/06; A61K 38/57; C12N 15/113

(52) U.S. Cl. CPC A61K 38/17; A61K 45/06; C12N 2310/14; C12N 15/1138; A61K 38/57

(57) ABSTRACT

The present invention features, in certain aspects, methods of promoting endocytosis of LDL with α1PI or peptides derived from α1PI. The present invention also provides methods for decreasing LDL levels in response to α₁PI augmentation therapy. In preferred embodiments, the methods are suitable for a subject who is suffering from a disease or disorder selected from heart disease, atherosclerosis, hypertension, HIV infection, viral infection, bacterial infection, leukemia, a solid tumor, or autoimmune disease.

### CROSS-REFERENCE TO RELATED APPLICATION

This application is a continuation of International Patent Application No. PCT/US2010/059664, filed on Dec. 9, 2010, which claims the benefit of U.S. Provisional Application No. 61/267,975, filed Dec. 9, 2009, the entire contents of which are incorporated herein by reference.

### RELATED APPLICATIONS/PATENTS & INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

Full length active α₁proteinase inhibitor (α₁PI, α₁antitrypsin) is composed of 394 amino acids (aa) having a mass of approximately 55 kDa when fully glycosylated (Berninger, 1985). Hepatocytes are the primary source of α₁PI, and in normal, healthy individuals, the range of circulating α₁PI is 20-5311M between the 5^{th} and 95^{th} percentiles (Brantly et al., 1991; Bristow et al., 1998). However, during the acute phase of the inflammatory response, α₁PI may increase as much as 4-fold to 200 μM (Kushner, 1982). There are four common alleles of α₁PI, and these are synthesized and secreted principally by hepatocytes (OMIM, 2000). However, there are more than a hundred genetic variants, some of which produce a molecule that prohibits secretion, and affected individuals manifest with 10-15% of the normal level of α₁PI in blood (Berninger, 1985). Individuals with this inherited form of α₁PI deficiency, especially males, are notably susceptible to respiratory infections and emphysema, and 80% who survive to adulthood succumb to respiratory failure between the fourth and sixth decades of life (Berninger, 1985). Prevalence is 0.03%, and α₁PI augmentation therapy in affected individuals is the only approved therapeutic application of α₁PI (OMIM, 2000).

Stem cell migration from Drosophila to humans requires LDL receptor-mediated Wnt-induced signaling (Cselenyi et al., 2008), and in humans requires α1 proteinase inhibitor (α1PI, α1antitrypsin, serpin A1) (Goselink et al., 1996), its receptor cell surface human leukocyte elastase (HLE-CS) (Bristow et al., 2003b; Lapidot and Petit, 2002), the chemokine CXCL12 (SDF-1), and its receptor CXCR4 (Lapidot and Petit, 2002), the same components involved in HIV-1 uptake (Bristow et al., 2003b). It has long been known that coupling of active α1PI to soluble HLE inactivates both proteins and exposes the C-terminal domain of α1PI (C-36, VIRIP) which then binds to receptors for low density lipoprotein (LDL) (Cao et al., 2006). Yet, it has not been appreciated that HLE is also localized on the cell surface (Bristow et al., 1995), and that when α1PI binds to HLE-CS at the leading edge of a migrating cell, the complex induces receptor polarization (Bristow et al., 2003b; Bristow et al., 2008e). Due to forward movement of the cell, receptor complexes underneath the cell reposition in “millipede-like locomotion” (Shulman et al., 2009) to the trailing edge where α1PI binds to LDL receptors, a condition that induces endocytosis of the complex and retraction of the trailing edge, and recycling of receptors to the leading edge of the migrating cell via endosomes in conveyor belt type motion (Cao et al., 2006).

If one of the components involved in this conveyor belt mechanism is missing or blocked, the cell halts migrating. For example, bacteria, snake bites, blood clotting, and most other non-normal situations produce non-normal proteases which cleave sentinel proteinase inhibitors including α1PI which is the most abundant proteinase inhibitor in serum. When α1PI is inactivated, it can no longer bind its receptor HLE-CS. In the absence of α1PI-HLE-CS complexes, the LDL receptors are not triggered for endocytosis and this causes blood cells to stop migrating.

Endocytosed LDL receptor-associated components are tagged for degradation, used as nutrients, or recycled to the cell surface from the trailing edge back to the leading edge via endosomes. In addition to dietary lipids, many nutrients such as insulin and growth factors are taken up by receptors that cluster with LDL receptors during the migration of granulocytes, macrophages, and lymphocytes which deliver and present endocytic cargo to the liver, mucosa, and other tissues. RAP is a 39 kDa exocytic traffic chaperone that binds LDL receptors thereby preventing their association with lipoproteins and proteinase inhibitors during synthesis. Endocytosed LDL Receptor-Associated Protein (RAP)-coupled LDL receptors dissociate in late endosomes (Czekay et al., 1997) where RAP is ubiquitinated and degraded, but LDL receptors are recycled (Misra and Pizzo, 2001). By contrast, when the LDL receptors are first bound to a proteinase inhibitor, it is the LDL receptors that are ubiquitinated. Thus, whether endo some components are tagged, degraded, recycled, or used as nutrients is determined by the order and context of LDL receptor-associated coupling during uptake and re-expression of selective cargo such as Wnt (Cselenyi et al., 2008), neurotransmitters (Andrade et al., 2007), antigenic material (Bristow and Flood, 1993; Robert et al., 2008), rhinovirus (Marlovits et al., 1998), Hepatitis C (Bartosch et al., 2003), and HIV-1 (Zhadina et al., 2007).

### SUMMARY OF THE INVENTION

The present invention is directed to the use of α₁PI and peptides derived from α₁PI to promote endocytosis of LDL. The present invention also provides methods for decreasing LDL levels in response to α₁PI augmentation therapy. In preferred embodiments, the methods are suitable for a subject who is suffering from a disease or disorder selected from heart disease, atherosclerosis, hypertension, HIV infection, viral infection, bacterial infection, leukemia, a solid tumor, or autoimmune disease. Under physiologic conditions proteinase inhibitors act as cogs in the LDL receptor-mediated cell migration apparatus and under pathologic conditions, inactivation by microbial proteinases or inflammation-induced cellular proteinases halts migration of selective cells specifically at the site of pathologic insult such as in atherosclerotic plaque, Alzheimer's fibrosis, chronic obstructive pulmonary disease, rheumatoid arthritis, multiple sclerosis, and tumor invasion.

In one aspect, the present invention provides a method of decreasing low density lipoprotein (LDL) levels in a subject comprising administering to the subject α1PI or at least one peptide derived from α1PI, thereby decreasing the levels of LDL in the subject.

In another aspect, the present invention features a method of modulating the distribution of LDL levels, HDL levels, cholesterol levels, triglyceride levels and other lipids derived from LDL, HDL, cholesterol, and triglycerides in a subject comprising administering to the subject α1PI or peptides derived from α1PI, thereby modulating the distribution of LDL levels, HDL levels, cholesterol levels, triglyceride levels and other lipids derived from LDL, HDL, cholesterol, and triglycerides in the subject.

In one embodiment, the α1PI peptides are produced by peptide synthesis or by recombinant plasmid transfection.

In another embodiment, the α1PI peptides that are used in the methods of the invention are derived from a wild-type amino acid selected from the group consisting of residues 370-374 and 385. In another embodiment, at least one amino acid selected from the group consisting of residues 370-374 and 385 is changed from wild-type to glycine, threonine, or a hydrophobic amino acid. In another embodiment, the hydrophobic amino acid is selected from the group consisting of isoleucine, leucine, phenylalanine, tyrosine and valine.

In certain embodiments, the α1PI peptides that are used in the methods of the invention comprise a change in a wild-type amino acid residues selected from the group consisting of residues 370-374 and 385. In another embodiment, the residue change is to glycine, threonine, or a hydrophobic amino acid. In another embodiment, the hydrophobic amino acid is selected from the group consisting of isoleucine, leucine, phenylalanine, tyrosine and valine. In another embodiment, the human α1PI peptides comprise at least two changes in wild-type amino acid residues selected from the group consisting of residues 370-374 and 385. In another embodiment, the methionine at position 385 is changed to a non-methionine amino acid. In another embodiment, the non-methionine amino acid is selected from the group consisting of glycine, isoleucine, leucine, phenylalanine, threonine, and valine. In another embodiment, the residue changes in the human α1PI peptides comprise the following three amino acid substitutions: Phe372Gly; Leu373Gly; and Met 385Val. In another embodiment, the residue changes in the human α1PI peptides consist of the following three amino acid substitutions: Phe372Gly; Leu373Gly; and Met 385Val.

In one embodiment, the subject is suffering from a disease or disorder selected from the group consisting of: heart disease, atherosclerosis, hypertension, HIV infection, viral infection, bacterial infection, leukemia, Alzheimer's Disease, a solid tumor, or autoimmune disease.

In another embodiment, the subject is suffering from HIV-1.

In a further embodiment, the number of CD4 T cells in the subject is less than 500 CD4 cells/μl.

In another embodiment, the subject is receiving proteinase inhibitor therapy.

In still another embodiment, α1PI or peptides derived from α1PI decrease LDL levels by promoting LDL endocytosis.

In another further embodiment, α1PI or peptides derived from α1PI decrease LDL levels by promoting LDL transport.

In a further related embodiment, the LDL receptor is very low density LDL (VLDL) receptor.

In another embodiment, the subject is a human or a non-human animal.

In another embodiment of the above aspects, the method further comprises administering a LDL inhibitor.

In another embodiment of the above aspects, the LDL inhibitor is a nucleic acid inhibitor, a small molecule inhibitor, a peptide or a peptide mimetic.

In a further embodiment, the nucleic acid inhibitor is a siRNA.

In another embodiment, the peptide or peptide mimetic comprises LDL Receptor Associated Protein.

The invention provides a method of treating a subject with a disease associated with an increase in the level of LDL, comprising: identifying a subject in need of treatment; administering to the subject α1PI or at least one peptide derived from α1PI; determining the level of LDL in the subject; wherein, following the administration, there is a decrease in the level of LDL in the subject, thereby treating the disease.

The invention also provides a method of treating a subject with a disease associated with an increase in the level of LDL, comprising; administering to the subject α1PI or at least one peptide derived from α1PI identified as capable of decreasing the level of LDL in the subject; determining the level of LDL in the subject; wherein following the administration, there is a decrease in the level of LDL in the subject thereby treating the disease.

The invention also provides a method of treating a subject with a disease, comprising; administering to the subject α1PI or at least one peptide derived from α1PI identified as capable of decreasing the level of LDL in the subject wherein following the administration, there is a decrease in the level of LDL in the subject thereby treating the disease.

The invention also provides a method of monitoring the treatment of a subject diagnosed with a disease associated with an increase in the level of LDL levels comprising: administering to the subject α1PI or at least one peptide derived from α1PI; and

comparing the level of LDL of the subject before and after administration of the α1PI or at least one peptide derived from α1PI.

In one embodiment, following administration of the α1PI or at least one peptide derived from α1PI there is a decrease in the level of LDL in the subject thereby indicating treatment.

The invention also provides a method of monitoring the treatment of a subject diagnosed with a disease associated with an increase in the level of LDL comprising: determining the level of LDL in the subject; administering to the subject α1PI or at least one peptide derived from α1PI; and comparing the level of LDL of the subject with the level of LDL of a control subject that is not diagnosed with the disease.

In one embodiment, following administration of α1PI or at least one peptide derived from α1PI there is a decrease in the level of LDL of the subject diagnosed with the disease as compared to the control subject, thereby indicating treatment.

The invention also provides a method of treating a subject with a disease associated with an increase in the level of LDL comprising: administering to the subject α1PI or at least one peptide derived from α1PI; and determining the level of LDL; wherein following the administration there is a decrease in the level of LDL thereby treating the disease.

The invention also provides a method of decreasing the level of LDL in a subject comprising: contacting a cell with α1PI or at least one peptide derived from α1PI; and determining the level of LDL of the subject; wherein the level of LDL decreases following the contact.

The invention also provides a method of designing a treatment protocol for a subject diagnosed with a disease associated with an increase in the level of LDL; comprising determining the level of LDL in the subject diagnosed with the disease; and comparing the level of LDL in the subject with the level of LDL of a control subject that does not have the disease;

wherein an increase in the level of LDL of the subject as compared to the control indicates that α1PI or at least one peptide derived from α1PI that is identified as capable of decreasing the level of LDL of the subject should be administered to the subject; and wherein no increase in the level of LDL in the subject as compared to the control indicates that α1PI or at least one peptide derived from α1PI that is identified as capable of decreasing the level of LDL of the subject should not be administered to the subject.

In one embodiment of any of the methods described herein, α1PI or at least one peptide derived from α1PI is administered in a therapeutically effective amount or a pharmaceutically acceptable salt or prodrug thereof, or a pharmaceutical composition comprising a therapeutically effective amount or a pharmaceutically acceptable salt or prodrug thereof, to the subject, thereby treating the disease.

In one embodiment, the methods of the invention further comprise obtaining α1PI or at least one peptide derived from α1PI or the pharmaceutically acceptable salt or prodrug thereof.

In another embodiment, the subject is a mammal.

In another embodiment, the subject is a human.

In another embodiment, the therapeutically effective amount is in the range of 5-100 μM.

In another embodiment, the therapeutically effective amount of the α1PI or at least one peptide derived from α1PI is administered by topical application, intravenous drip or injection, subcutaneous, intramuscular, intraperitoneal, intracranial and spinal injection, ingestion via oral route, inhalation, trans-epithelial diffusion or an implantable, time-release drug delivery device.

In another embodiment, the results of the determining step are reported to the subject and/or a health care professional.

The invention also provides for a packaged pharmaceutical comprising α1PI or at least one peptide derived from α1PI or a pharmaceutically acceptable salt or prodrug thereof which, upon administration to a subject, decreases the level of LDL of a subject.

The invention also provides for a packaged pharmaceutical comprising

(a) α1PI or at least one peptide derived from α1PI or a pharmaceutically acceptable salt or prodrug thereof; and

(b) associated instructions for using the α1PI or at least one peptide derived from α1P1 to treat a disease associated with an increase in the level of LDL of a subject.

In one embodiment, the α1PI or at least one peptide derived from α1PI is present as a pharmaceutical composition comprising a therapeutically effective amount or a pharmaceutically acceptable salt or prodrug thereof and a pharmaceutically acceptable carrier.

In one embodiment, the packaged pharmaceutical further comprises a step of identifying a subject in need of the pharmaceutical.

The packaged pharmaceutical further comprises a step of identifying the α1PI or at least one peptide derived from α1PI as capable of decreasing the level of LDL in a subject.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 is a graph that shows the influence of α₁PI on serum lipid levels. (a) Lipid levels (mg/dL) are depicted by squares (▪) if receiving HIV-1 protease inhibitor therapy and by circles (●) if not. In patients with <500 CD4 cells/μl, LDL was correlated with active α₁PI (r²=0.44, p<0.001, n=24). (b) LDL exhibited negative correlation in patients with <20 μM inactive α₁PI (r²=0.35, p<0.001, n=33) and positive correlation in patients with >20 μM inactive α₁PI (r²=0.29, p<0.02, n=19).

FIG. 2 is a graph that shows the kinetic influence of α₁PI on HIV binding. (a) As compared with buffer (purple) or isotype control (pink), flow cytometric analysis depicts SHIV or SIV (green) bound to U937 clone 10 cells that had been preconditioned with α₁PI for 15 min (t₁₅), but not 60 min (t₆₀). (b) Fluorescence microscopic examination of clone 10 and human (c) immature and (d) mature MDC depicts virus (green) and cells using nuclear staining DAPI (blue). (e) Clone 10 cells were preconditioned with active α₁PI for 0 min (▾), 15 min (⋄), or 60 min (●) prior to addition of infectious HIV-1 NL4-3. Negative control cells (▪) were incubated with α₁PI for 15 min in the presence of the HIV-1 fusion inhibitor T20. Cells were prepared 3 times, and representative data are presented. Cells cultured in autologous sera with and without exogenous α₁PI (f) during (t₀) or (g) 60 min (t₆₀) prior to in vitro co-culture with a CCR5-using primary isolate of HIV-1. Influence of α₁PI on HIV outcome is represented as ΔHIV=100*(HIV spiked with α₁PI)/(HIV without spiking). HLE_{CS} mean fluorescence intensity (MFI) on monocytic cells (CD14±) correlated with ΔHIV at t₀ (r²=0.87, n=6, p=0.006) and t₆₀ (r²=0.96, n=4, p=0.02). Active α₁PI was correlated with ΔHIV (r²=0.95, p=0.001, n=6) at t₆₀, but not t₀. PBMC and sera from different volunteers were examined at least 3 times, and representative data are presented.

FIG. 3 is a graph that shows the influence of VLDLR on receptor recycling and on HIV-1 uptake. (a) By flow cytometric analysis, U937 clone 10 transfected with VLDLR siRNA (green) expressed 57% less VLDLR, 44% more CD4, 100% more CXCR4, and 10% less HLE_{CS} than cells transfected with negative control siRNA (red). (b) HIV-1 infectivity of cells from part (a) transfected with VLDLR siRNA (●) or with negative control siRNA (◯). (c) By confocal microscopic analysis of day 0 cells from part (b), cells (depicted by differential interference contrast) did not internalize HIV-1 (green) after transfection with VLDLR siRNA. Bar represents 25 μm. (d) HIV-1 infectivity of clone 10 in the absence of RAP and α₁PI (●), after preconditioning for 15 min with α₁PI (◯), and after preconditioning with RAP for 15 min followed by 15 min with α₁PI (▴). Cells were prepared at least three times, and representative results are presented.

FIG. 4 demonstrates feedback regulation by LDL and α₁PI. LDL levels decreased in two HIV-1 patients Alpha (a) and Beta (b) placed on α₁PI augmentation therapy (Zemaira®, CSL Behring) (Table S2). In patient Alpha, decreased LDL was correlated increased active α₁PI Alpha (r²=0.61, n=9, p=) and decreased inactive α₁PI (r²=0.61, n=9, p=). In patient Beta, decreased LDL was correlated increased active α₁PI Alpha (r²=0.42, n=6, p=) and with decreased inactive α₁PI (r²=0.90, n=6, (c) DNA microarray analysis of adherent cells from a healthy volunteer and HIV patients on HIV protease inhibitor therapy (ritonavir). Gene expression ratio of patient to healthy cells was calculated using data from 2 patients. All the genes known to have lipoprotein and proteinase inhibitor function that changed more than 10-fold are represented. Probe sets ending with x_at and s_at, were deleted.

FIG. 5 demonstrates the expression of VLDLR but not LRP on U937 clone 10 cells. By flow cytometric analysis, as compared to isotype control (▪), CD91 (▪) was not detected on (a) U937 clone 10 cells, but was detected on (b) primary monocytic cells. (c) As compared to 10 μM nonspecific siRNA, there was dose-dependent loss of VLDLR expression 48 hrs after transfection of cells with ( [symbol] ) 0.05 μM ( [symbol] ), 0.1 μM ( [symbol] ), 1 (▪), and 10 μM (▪) VLDLR siRNA. The ratio VLDLR siRNA/nonspecific siRNA yielded VLDLR expression of 100%, 76%, 52%, 43%, and 31% for respective doses of VLDLR siRNA.

### DETAILED DESCRIPTION OF THE INVENTION

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used in the specification and claims, the singular form “a”, “an” and “the” include plural references unless the context clearly dictates otherwise.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The terms “administration” or “administering” are defined to include an act of providing a compound or pharmaceutical composition of the invention to a subject in need of treatment. In the instant invention, preferred routes of administration include parenteral administration, preferably, for example by injection, for example by intravenous injection.

As used herein, the term “control” is meant a standard or reference condition.

The term “active α1PI” as used herein is meant to refer to the fraction of α1PI in plasma or other fluids that has the capacity to inhibit elastase activity.

The term “inactive α1PI” as used herein is meant to refer to the fraction of α1PI in plasma or other fluids that does not have the capacity to inhibit elastase activity. Active α1PI may be inactivated by proteolytic cleavage, proteinase complexing, antibody complexing, or oxidation.

The term “human immunodeficiency virus” or HIV is meant to refer to a virus that consists of either an HIV-1 or an HIV-2 virus, and more particularly any virus strain or isolate of an HIV-1 or an HIV-2 virus.

As used herein, the term “alpha1-Proteinase Inhibitor” (α1PI) is meant to refer to a glycoprotein produced by the liver and secreted into the circulatory system. α1PI belongs to the Serine Proteinase Inhibitor (Serpin) family of proteolytic inhibitors. This glycoprotein of MW of 50,600 Da consists of a single polypeptide chain containing one cysteine residue and 12-13% carbohydrates of the total molecular weight. α1PI has three N-glycosylation sites at asparagine residues 46, 83 and 247, which are occupied by mixtures of complex bi- and triantennary glycans. This gives rise to multiple α1PI isoforms, having isoelectric point in the range of 4.0 to 5.0. The glycan monosaccharides include N-acetylglucosamine, mannose, galactose, fucose and sialic acid. α1PI serves as a pseudo-substrate for elastase; elastase attacks the reactive center loop of the α1PI molecule by cleaving the bond between methionine358-serine359 residues to form an α1PI-elastase complex. This complex is rapidly removed from the blood circulation. α1PI is also referred to as “alpha-1 antitrypsin” (AAT). In certain embodiments, α1PI is human α1PI and is encoded by the amino acid sequence set forth by NCBI Accession No. KO1396.

As used herein, the term “subject” is intended to include vertebrates, preferably a mammal. Mammals include, but are not limited to, humans.

As used herein, the term “peptide” means a polymer of amino acids linked via peptide bonds. A peptide according to the invention can be two or more amino acids, for example, 2, 3, 4, 5, 6, 7, 8, 9, 10, 15, 20 or more.

As used herein the term “at least one peptide” means one or more (for example, 1, 2, 3, 4, 5, 6, 7, 8, 9, 10, 15, 20 or more) peptides derived from α1PI.

“Treatment”, or “treating” as used herein, is defined as the application or administration of α1PI or at least one peptide derived therefrom to a subject or patient, or application or administration of α1PI or at least one peptide derived therefrom to an isolated tissue or cell line from a subject or patient, who has a disease or disorder that is associated with an increased level of LDL, with the purpose to cure, heal, alleviate, relieve, alter, remedy, ameliorate, improve or affect the disease or disorder, or symptoms of the disease or disorder. The term “treatment” or “treating” is also used herein in the context of administering agents prophylactically. The term “effective dose” or “effective amount” or “effective dosage” or “therapeutic dosage” is defined as an amount sufficient to achieve or at least partially achieve the desired effect. The terms “therapeutically effective dose” and “therapeutically effective amount” are defined as an amount sufficient to cure or at least partially arrest the disease and its complications in a patient already suffering from the disease.

As used herein, “patient” or “subject” refers to a mammal that is diagnosed with a disease associated with an increase in the level of LDL.

The term “patient” or “subject” includes human and other mammalian subjects that receive either prophylactic or therapeutic treatment.

As used herein, “mammal” refers to any mammal including but not limited to human, mouse, rat, sheep, monkey, goat, rabbit, hamster, horse, cow or pig.

A “non-human mammal”, as used herein, refers to any mammal that is not a human.

As used herein, “treating” a disease refers to preventing the onset of disease and/or reducing, delaying, or eliminating disease symptoms, such as an increase in the level of LDL. By “treating” is meant restoring the patient or subject to the basal state as defined herein, and/or to prevent a disease in a subject at risk thereof. Alternatively, “treating” means arresting or otherwise ameliorating symptoms of a disease.

As used herein, “basal state” refers to the level of LDL of an individual who is not susceptible to a disease and who has no symptoms of a disease and/or an individual who has not been diagnosed with the disease.

In one embodiment, a disease according to the invention is associated with an increase in the level of LDL in a subject.

As used herein, “diagnosing” or “identifying a patient or subject having” refers to a process of determining if an individual is afflicted with a disease or ailment, for example a disease associated with an increase in the level of LDL. To diagnose a disease associated with an increase in the level of LDL the level of LDL is measured by methods known in the art including but not limited to methods described hereinbelow.

As used herein, “modulate” or “modulation” refers to increase or decrease, or an increase or a decrease, for example an increase or decrease in the level of LDL.

As used herein, “decrease” means that the level of LDL is 1, 2, 3, 4, 5, 10, 15, 20, 25, 30, 35, 40, 45, 50, 100, 500, 1000 or 10,000-fold less after administration of α1PI or at least one peptide derived therefrom as compared to before administration of α1PI or at least one peptide derived therefrom of the invention.

As used herein, “decrease” also means that the level of LDL is 1, 5, 10, 15, 20, 25, 30, 35, 40, 45, 50, 55, 60, 65, 70, 75, 80, 85, 90, 95, 99 or 100% less after administration of α1PI or at least one peptide derived therefrom of the invention as compared to before administration of α1PI or at least one peptide derived therefrom of the invention.

As used herein “increased” as it refers to level of LDL, means that the level of LDL is 1, 2, 3, 4, 5, 10, 15, 20, 25, 30, 35, 40, 45, 50, 100, 500, 1000 or 10,000-fold or more greater in a patient diagnosed with a disease associated with an increase in the level of LDL as compared to a control subject that is not diagnosed with the disease.

As used herein “increased” as it refers to the level of LDL, means that the level of LDL is 1, 5, 10, 15, 20, 25, 30, 35, 40, 45, 50, 55, 60, 65, 70, 75, 80, 85, 90, 95, 99 or 100% greater in a patient diagnosed with a disease associated with an increase in the level of LDL as compared to a control subject that is not diagnosed with the disease.

As used herein, “increase” means that the level of LDL is 1, 2, 3, 4, 5, 10, 15, 20, 25, 30, 35, 40, 45, 50, 100, 500, 1000 or 10,000-fold more after administration of α1PI or at least one peptide derived therefrom of the invention as compared to before α 1PI or at least one peptide derived therefrom of the invention.

As used herein, “increase” also means that the level of LDL is 1, 5, 10, 15, 20, 25, 30, 35, 40, 45, 50, 55, 60, 65, 70, 75, 80, 85, 90, 95, 99 or 100% more after administration of α1PI or at least one peptide derived therefrom of the invention as compared to before administration of α1PI or at least one peptide derived therefrom of the invention

A method of “administration” useful according to the invention includes but is not limited to subcutaneous, intramuscular, intraperitoneal, intracranial and spinal injection, ingestion via the oral route, inhalation, trans-epithelial diffusion (such as via a drug-impregnated, adhesive patch), by the use of an implantable, time-release drug delivery device, which may comprise a reservoir of exogenously-produced agent or may, instead, comprise cells that produce and secrete the therapeutic agent or topical application or administration directly to a blood vessel, including artery, vein or capillary, intravenous drip or injection. Additional methods of administration are

A “therapeutically effective amount” of α1PI or at least one peptide derived therefrom of the invention according to the invention is in the range of 5-100 μM subject. In another embodiment, a “therapeutically effective amount” of α 1PI or at least one peptide derived therefrom is in the range of 10-75 μM per subject. In another embodiment, a “therapeutically effective amount” of α 1PI or at least one peptide derived therefrom is in the range of 20-50 μM per subject. In another embodiment, a “therapeutically effective amount” of α 1PI or at least one peptide derived therefrom is in the range of 18-48 μM per subject. In another embodiment, a “therapeutically effective amount” of α 1PI or at least one peptide derived therefrom is in the range of 5-25 μM per subject. In another embodiment, a “therapeutically effective amount” of α 1PI or at least one peptide derived therefrom is in the range of 5-20 μM per subject. In another embodiment, a “therapeutically effective amount” of α 1PI or at least one peptide derived therefrom is in the range of 5-10 μM per subject.

As used herein “monitoring the treatment” means determining whether, following treatment of a subject, for example, a subject diagnosed with a disease associated with an increase in the level of LDL, the subject has been treated such that the symptoms of the disease are arrested or otherwise ameliorated and/or the disease and/or its attendant symptoms are alleviated or abated.

As used herein, “control subject” means a subject that has not been diagnosed with a disease and/or does not exhibit any detectable symptoms associated with the disease, for example a disease associated with an increase in the level of LDL.

As used herein, the term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

“Prodrug”, as used herein means a compound which is convertible in vivo by metabolic means (e.g. by hydrolysis) to afford any compound delineated by the formulae of the instant invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

Other definitions appear in context throughout the disclosure.

Treatment Population:

Active α₁PI promotes migration of lymphocytes and monocytic cells expressing HLE_{CS} (Bristow et al., 2003a; Bristow et al., 2008d). Inactive α₁PI promotes migration of neutrophils and cells expressing the LDL receptors including LDL-receptor related protein, LRP, LDL receptor, and VLDL receptor (Kounnas et al., 1996; Weaver et al., 1997). Treatment with human α₁PI is indicated in individuals manifesting abnormal distribution of LDL levels, HDL levels, cholesterol levels, triglyceride levels and other lipids derived from LDL, HDL, cholesterol, and triglycerides manifested in a disease, disorder or condition selected from the group consisting of heart disease, atherosclerosis, hypertension, HIV infection, viral infection, bacterial infection, leukemia, a solid tumor, and autoimmune disease. Treatment outcome is determined as described below in Section 7 of the Detailed Description.

### Treatment Regimen:

The dosage of an α₁PI peptide is determined by its capacity to promote endocytosis of LDL as described in Section 6 of the Detailed Description. Individuals are injected with α₁PI peptides in the concentration range of 5-100 μM. The frequency and length of treatment are determined by the decrease in LDL levels. In addition to being monitored for PPE inhibitory activity, α₁PI peptides are screened as described in Section 3.2 of the Detailed Description for their capacity to induce receptor capping and cell motility of lymphoid- and myeloid-lineage blood cells such as lymphocytes and neutrophils.

### Recombinant α₁PI:

In addition to α₁PI peptides synthesized from individual amino acids, recombinant α₁PI peptides will be used to promote decreased LDL levels.

Structural Features of α₁PI:

The following represents the full length amino acid sequence for α₁PI (accession # K01396) including the 24 aa signal peptide:

```
−24 MPSSVSWGIL LLAGLCCLVP VSLA
    1 EDPQGDAAQK TDTSHHDQDH PTFNKITPNL AEFAFSLYRQ LAHQSNSTNI
  51 FFSPVSIATA FAMLSLGTKA DTHDEILEGL NFNLTEIPEA QIHEGFQELL
101 RTLNQPDSQL QLTTGNGLFL SEGLKLVDKF LED VKKLYHS EAFTVNFGDT
151 EEAKKQINDY VEKGTQGKIV DLVKELDRDT VFALVNYIFF KGKWERPFEV
201 KDTEEEDFHV DQVTTVKVPM MKRLGMFNIQ HCKKLSSWVL LMKYLGNATA
251 IFFLPDEGKL QHLENELTHD IITKFLENED RRSASLHLPK LSITGTYDLK
301 SVLGQLGITK VFSNGADLSG VTEEAPLKLS KAVHKAVLTI DEKGTEAAGA
351 MFLEAIPMSI PPEVKFNKPF VFLMIEQNTK SPLFMGKVVN PTQK
```

The known Asn-linked carboxylation sites (denoted in bold underlined letters) are found at aa 46, 83, and 247 (Nukiwa et al., 1986; Jeppsson et al., 1985). The oligosaccharide structure at each site is either tri-antenary or bi-antenary, and the various combinations give the protein a characteristic electrophoretic charge denoted as phenotypic subtypes of the four common genotypic alleles, M1A, M1V, M2, and M3. The frequencies in US Caucasians of M1A, M1V, M2, and M3 are 0.20-0.23, 0.44-0.49, 0.1-0.11, and 0.14-0.19, respectively, accounting for 95% of this population (Jeppsson et al., 1985). M1A is thought to be the oldest variant, and M1V has a single aa substitution, at position 213, Ala to Val. The M3 allele has a single aa difference with M1V, Glu to Asp at position 376. The M2 allele has a single aa difference with M3, Arg to His at position 101.

More than a hundred genotypic alleles have been identified, but except for the S and Z alleles, most of them are exceedingly rare (OMIM, 2000). The S allele, frequency 0.02-0.04, has a single aa substitution at position 264, Glu to Val, and individuals homozygous for this allele manifest 60% normal α₁PI blood levels, but are not at risk for emphysema or other known diseases except in combination with the Z allele (Brantly et al., 1991; Sifers et al., 1988). The Z allele, frequency 0.01-0.02, has a single aa substitution at position 342, Glu to Lys, and individuals homozygous for this allele manifest 10% normal α₁PI blood levels, and are at risk for emphysema and autoimmunity.

Functional Properties of α₁PI:

There are three distinct activities of α₁PI that are determined by sites in the C-terminal region of α₁PI defined herein as aa 357-394,

PMSI PPEVKFNKPF VFLMIEQNTK SPLFMGKVVN PTQK.

The crystal structure for active α₁PI (1HP7, NIH NCBI Molecular Modeling DataBase mmdbld:15959) has been determined. The C-terminal region of α₁PI (aa 369-394) forms a β-sheet. Two α-helix domains (aa 27-44 and 257-280) shield the β-sheet domain in a manner resembling the antigen-binding cleft of the major histocompatibility complex.

The first activity of α₁PI is its well characterized proteinase inhibition which is a property only of active, uncleaved α₁PI. The reactive site for this activity is Met (aa 358) contained in the domain Pro-Met-Ser-Ile-Pro (PMSIP, aa 357-361). Active α₁PI may be inactivated by proteinase complexing, cleavage, or oxidation of Met (aa 358). Interaction at the scissile bond Met-Ser (aa 358-359) may be mediated by many proteinases including HLE_{G}. The two cleavage products of α₁PI may dissociate under some circumstances, but may remain associated in a new, rearranged configuration that may irreversibly incorporate HLE_{G}, but may not incorporate other proteinases, for example metalloproteinases (Perkins et al., 1992).

The tertiary structure for the rearranged α₁PI configuration has not been solved (Mellet et al., 1998); however, X-ray diffraction and kinetic analyses of cleaved α₁PI suggest that the strand SIPPEVKFNKP (aa 359-369) may separate 70A° from its original position and insert into the β-sheet formation on the opposite face of the molecule ((3-sheet A) in a manner that would significantly alter proteinase and receptor recognition (Elliott et al., 2000). Thus, four configurations of the C-terminal region of α₁PI are thought to occur (Table 1).

TABLE 1
Functions of the C-terminal region of α₁PI

| | Proteinase | Lymphoid cell | Myeloid cell |
|---|---|---|---|
| | Inhibition | migration | migration |
| Native configuration in | + | + | − |
| the active α₁PI | | | |
| Rearranged configuration in | − | − | Unknown |
| cleaved α₁PI | | | |
| Complexed with HLE in | − | + | + |
| inactivated α₁PI | | | |
| Independent of other α₁PI | − | Unknown | + |
| cleavage products | | | |

Because the cleaved configuration of α₁PI lacks proteinase inhibitory activity, in deficient concentrations of active α₁PI, the result is emphysema and respiratory-related infections which are facilitated by the presence of certain environmental factors, cigarette smoke, microbial factors, and inherited mutations that prohibit successful production of active α₁PI.

A second activity of α₁PI is the stimulation of cell migration. It has long been known that coupling of active α₁PI to soluble granule-released HLE (HLE_{G}) inactivates both proteins and exposes the C-terminal domain of α₁PI (C-36, VIRIP) which then binds to receptors for low density lipoprotein (LDL) (Janciauskiene et al., 2001). Yet, it has not been appreciated that HLE is also localized on the cell surface (Bangalore and Travis, 1994) and that when α₁PI binds to HLE_{CS} at the leading edge of a migrating cell, due to forward movement of the cell, the receptor complexes underneath the cell reposition in “millipede-like locomotion” (Shulman et al., 2009) to the trailing edge where α₁PI binds to LDL receptors at the trailing edge of the same cell, a condition that induces endocytosis of the receptor aggregate and retraction of the trailing edge of the cell (Cao et al., 2006). Endocytosed LDL receptor-associated components are tagged for degradation, used as nutrients, or recycled to the cell surface from the trailing edge back to the leading edge via endosomes. In addition to dietary lipids, many nutrients such as insulin and growth factors are taken up by receptors that cluster with LDL receptors during the migration of granulocytes, macrophages, and lymphocytes which deliver and present endocytic cargo to the liver, mucosa, and other tissues.

Expression of Recombinant α₁PI Peptides:

Any method known in the art may be used for producing recombinant α₁PI peptides according to the invention. Two preferred methods are briefly described below for producing recombinant α₁PI peptides; one allows expression of α₁PI peptides in rice cells and the other allows bacterial expression. The cDNA encoding human α₁PI is obtained from a human cDNA bank and amplification of the fragment in accession number K01396 using two PCR primers: N-terminal primer 5′ GAGGATCCCCAGGGAGATGCTGCCCAGAA 3′ and C-terminal primer 5′CGCGCTCGAGTTATTTTTGGGTGGGATTCACCAC 3′ as previously described (Courtney et al., 1984; Terashima et al., 1999; Jean et al., 1998).

For expression in rice cells, expression cassettes are prepared by using a 1.1 kb NheI-PstI fragment, derived from p1AS1.5, and cloned into the vector pGEM5zf-(Promega, Madison, Wis.): ApaI, AatII, SphI, NcoI, SstII, EcoRV, SpeI, NotI, PstI, SalI, NdeI, SacI, MluI, NsiI at the SpeI and PstI sites to form pGEM5zf-(3D/NheI-PstI). The GEM5zf-(3D/NheI-PstI) is digested with PstI and SacI and ligated in two nonkinased 30mers with the complementary sequences 5′ GCTTG ACCTG TAACT CGGGC CAGGC GAGCT 3′ and 5′ CGCCT AGCCC GAGTT ACAGG TCAAG CAGCT 3′ to form p3DProSig. A 5-kb BamHI-KpnI fragment from lambda clone λOSg1 A is used as a terminator. Hygromycin resistance is obtained from the 3-kb BamHI fragment containing the 35S promoter-Hph-NOS of the plasmid pMON410.

Microprojectile bombardment is applied for transforming a Japonica rice variety TP309. The bombarded calli are then transferred to NB medium containing 50 mg/l hygromycin and incubated in the dark at 25° C. for 10±14 days. Rice cells are cultured at 28° C. (dark) using a shaker with rotation speed 115 rpm in the AA (+sucrose) media. The medium is changed every 5 days to maintain cell lines. AA (−sucrose) is used for α₁PI expression. A bioreactor is used for 2-1-scale culture. The reactor is operated at 28° C. (dark) at agitation speed 30±50 rpm with aeration rate 100 ml/min. During the growth phase (10 days), the pH of the media is controlled at pH 5.7, while in the production phase the pH is 5.7±6.3 (un-controlled).

Recombinant α₁PI peptides are purified using anti-human α₁PI antibody (Enzyme Research Laboratories, South Bend, Ind.) immobilized to CNBr-activated Sepharose 4B with a concentration of 1.5 mg/ml gel. The gel (3.5 ml) is packed in a column (inner diameter 1.26 cm), and equilibrated with 50 mM Tris-HCl buffer (pH 7.6). Crude medium is applied to the column at 1.0 ml/min. Absorbance at 280 nm is monitored at the outlet of the column. After washing with the equilibrium buffer, α₁PI is eluted with 0.1N HCl solution. A peak fraction is collected, and its pH is immediately adjusted with 1M Tris-HCl buffer (pH 8.0). These methods yield an estimated 5.7 mg α₁PI peptide/g dry cell.

Alternatively, the α₁PI peptides cDNA are expressed in Escherichia coli strain BL21 transformed with pDS56α₁PI/hf (Invitrogen, Carlsbad, Calif.). Protein expression is induced by addition of 1 mM isopropyl b-D-thiogalactoside, and cultures are grown overnight at 31° C. The cells are washed in metal-chelation chromatography binding buffer (5 mM imidazole/0.5M NaCl/20 mM Tris, pH 7.9) and disrupted by cavitation. The clarified and filtered supernatants containing soluble α₁PI peptides are applied to a Ni²⁺-agarose column, and bound peptides are eluted with 100 mM EDTA. The eluates are adjusted to 3.5M NaCl and applied to a phenyl-Sepharose column. The bound α₁PI peptide/hf is eluted with 20 mM Bis-Tris, pH 7.0 and concentrated (4 mg/ml final) by diafiltration in the same buffer.

### Genetic Modification of α₁PI Peptides:

Recombinant α1PI peptides are expressed according to the procedures described in Section 3 of the Detailed Description. Wild-type human α₁PI peptides are modified genetically to diminish or enhance sequence-specific reactive sites. For example, in HIV-1 disease, therapeutic α1PI peptides maintain binding to anti-gp120, but do not interfere with full sequence α₁PI in its activities to inhibit soluble HLE_{G} and to induce cell migration.

The genetic modifications of interest are described in Section 4.1 of the Detailed Description. Site-directed mutagenesis of active α1PI is performed using standard procedures (Parfrey et al., 2003; Current Protocols in Molecular Biology, 2002). The DNA sequence encoding the human α1PI signal peptide in pDS56α₁PI/hf is replaced with sequences encoding the epitope (FLAG)-tag by insertion of the annealed complimentary oligos 5′ CTAGAGGATCCCATGGACTACAAGGACGACGATGACAAGGAA 3′ and 5′GATCTTCCTTGTCATCGTCGTCCTTGTAGTCCATGGGATCCT 3′. The resulting cDNA is subcloned into pDS56-6His to generate pDS56α1PI/hf. To generate pDS56α1PI/hf carrying an amino acid substitution, the DNA sequence encoding the wild-type amino acids is replaced by the complimentary oligos coding for the amino acids described in Section 4.1 of the Detailed Description. The resulting ORFs directed cytosolic expression of the recombinant proteins initiating with a Met followed by the His and FLAG tags and the mature sequences of mutant α1PI.

Modification within the domain that determines cell migration (FVFLM, aa 370-374) is prepared by site-directed mutagenesis of specific amino acids:
Phe (aa 370) to Be, Leu, Val, Tyr, or Gly.
Val (aa 371) to Phe, Leu, Be, or Gly.
Phe (aa 372) to Be, Leu, Val, Tyr or Gly.
Leu (aa 373) to Ile, Val, Phe, or Gly.
Met (aa 374) to Phe, Thr, Ile, Leu, Val, or Gly.

Modification within the domain that determines LDL receptor recognition is prepared by site-directed mutagenesis of Met (aa 385) to Phe, Thr, Ile, Leu, Val, or Gly.

### 5. α₁PI Peptide Synthesis:

All peptides will be prepared by Fmoc solid-phase synthesis as previously described (Fields and Noble, 1990) and subsequently purified by reversed-phase chromatography. Identity and homogeneity of the products will be analyzed by reversed-phase HPLC, capillary zone electrophoresis, electrospray mass spectrometry, and sequence analysis. After proteolytic modification, the C-terminal α₁PI domain acquires attributes that allow interaction with the LDL receptor-related protein (LRP) (Poller et al., 1995), the VLDL receptor (Rodenburg et al., 1998), and other receptors that recognize a pentapeptide sequence FVFLM (aa 370-374) (Joslin et al., 1992) in a manner that produces, chemotaxis, increased LDL binding to monocytes, upregulated LDL receptors, increased cytokine production, and α₁PI synthesis (Banda et al., 1988; Janciauskiene et al., 1999b; Janciauskiene et al., 1999a). It has been shown that fibrillar aggregates of the C-terminal fragment of α₁PI facilitate uptake of LDL by LRP on the hepatoblastoma cell line HepG2 (Janciauskiene and Lindgren, 1999), and these fragments participate in atherosclerosis (Dichtl et al., 2000).

### 6. α₁PI Peptides and Therapy:

The desired α₁PI peptides for treating disease are those that produce decreased LDL levels, but do not functionally interfere with the physiologic activity of α₁PI. Peptides derived from α₁PI are selected for use in treatment of specific blood cell diseases by determining their capacity in vitro and in vivo to influence the following functions in the following assays:

### 6.1 Inhibit Elastase:

The procedures for measuring the capacity of α₁PI to inhibit soluble forms of porcine pancreatic elastase (PPE) or HLE_{G} are well established (U.S. Pat. No. 6,887,678) (Bristow et al., 1998). Briefly, PPE is incubated for 2 min with α₁PI, and to this mixture is added, the elastase substrate succinyl-L-Ala-L-Ala-L-Ala-p-nitroanilide (SA³NA). Results are detected by measuring the color change at 405 nm.

In complex mixtures, α₁PI competes for binding to PPE with other proteinase inhibitors or ligands present in the mixture. For example, PPE has higher affinity for α₂macroglobulin (α₂M) than for α₁PI, and when complexed with α₂M, PPE retains the ability to cleave small substrates. In the presence of α₂M, PPE binds α₂M and is protected from inhibition by α₁PI, and the complexation of PPE with α₂M can be measured by detecting the activity of PPE using SA³NA. To measure the inhibitory capacity of α₁PI in complex mixtures such as serum, two-fold serial dilutions of serum are incubated with a constant, saturating concentration of PPE. The added PPE is bound by α₂M and α₁PI in the diluted serum depending on their concentrations. The greater the concentration of serum, the greater the concentration of α₂M and α₁PI. Since there is more α₁PI in serum than α₂M, as serum is diluted, α₂M is diluted out, and in the absence of α₂M, PPE is bound and inhibited by α₁PI. The complexation of PPE with α₁PI can be measured by detecting the loss of activity of PPE using SA³NA. As serum is further diluted, α₁PI is also diluted out, and the loss of complexation of PPE with α₁PI can be measured by detecting the gain in activity of PPE using SA³NA. The plot of PPE activity versus serum dilution makes a V shaped curve, PPE activity first decreasing as serum is diluted, and then increasing as serum is further diluted. The nadir of PPE activity is used to calculate the precise concentration of active α₁PI in the mixture (Bristow et al., 1998).

### 6.2 Induce Receptor Co-Capping and Cell Motility:

The procedures for inducing receptor capping have been described (Bristow et al., 2003a; Bristow et al., 2008c). The cells of interest (monocytes, lymphocytes, neutrophils, or other blood cells, e.g. leukemic cells) are isolated from blood or tissue using standard techniques (Messmer et al., 2002b) and examined for reactivity with α₁PI.

To examine receptor capping, cells are incubated with active or modified α₁PI for 15 min in humidified 5% CO₂ at 37° C. Cells are applied to the sample chambers of a cytospin apparatus (Shandon Inc. Pittsburgh, Pa.), and slides are centrifuged at 850 rpm for 3 min. Slides are fixed by application of 50 μl 10% formalin to the sample chambers of the cytospin apparatus followed by an additional centrifugation at 850 rpm for 5 min. Slides are incubated for 90 min at 20° C. with fluorescently-labeled monoclonal antibodies having specificity for the receptors of interest and examined by microscopy.

Cell motility results from selective and sequential adherence and release produced by activation and deactivation of receptors (Wright and Meyer, 1986; Ali et al., 1996), consequent polar segregation of related membrane proteins to the leading edge or trailing uropod, and both clockwise and counterclockwise propagation of Ca⁺⁺ waves which initiate from different locations in the cell (Kindzelskii and Petty, 2003). Thus, several aspects of the complex process may be quantitated. The most direct and most easily interpreted method for quantitating cell motility is the enumeration of adherent cells in response to a chemotactic agent such as α₁PI.

For detecting adherence, sterile coverslips are washed in endotoxin-free water, and to each coverslip is delivered various dilutions of active or modified α₁PI. Cells are subsequently delivered to the coverslips, mixed to uniformity with α₁PI, and incubated for 30 min in humidified 5% CO₂ at 37° C. without dehydration. After stringently washing the coverslips free of non-adherent cells, adherent cells are fixed by incubation for 10 min at 20° C. with 4% paraformaldehyde containing 2.5 μM of the nuclear staining fluorescent dye, acridine orange (3,6-bis[dimethylamino]acridine. Slides are examined by microscopy, and means and standard deviations are determined by counting adherent cells in at least three fields/coverslip.

### 7. Treatment Outcome Measurements:

7.1 To determine the influence of α₁PI peptide treatment on elastase inhibitory capacity, individuals are monitored weekly for levels of active and inactive α₁PI in blood (Bristow et al., 1998) (U.S. Pat. No. 6,887,678). Briefly, a constant amount of active site-titrated PPE is allowed to incubate with serial dilutions of serum for 2 min at 37° C. after which a PPE substrate is added. Determination of the molecules of substrate cleaved by residual, uninhibited PPE is used to calculate the molecules of active and inactive α₁PI in blood.
7.2 To determine the influence of α₁PI peptide treatment on inducing changes in lipid levels of blood cell populations, treated individuals are monitored weekly for changes in complete blood count and differential, as well as for changes in LDL, HDL, cholesterol and triglycerides using standard, approved clinical laboratory methods.
7.3 To determine the influence of treatment on disease progression, individuals are monitored for the specific pathologic determinants of disease which are well known in the art for the various indications in heart disease, atherosclerosis, hypertension, HIV infection, viral infection, bacterial infection, leukemia, a solid tumor, and autoimmune disease.

### 8. Determining LDL Levels

Methods of determining the LDL level are known in the art.

LDL=Total cholesterol−HDL−(Triglycerides/5). Total cholesterol (mg/dL) is measured using an enzyme assay (cholesterol esterase/cholesterol oxidase) to produce hydrogen peroxide which causes a color change that can be quantitated (for example as described in Allain et al. 1974 Clin Chem 20:470-475.

HDL (mg/dL) is measured in a similar manner, in an assay utilizes an HDL specific detergent. The result is Total cholesterol+HDL.

Triglycerides (mg/dL) are measured using a different set of enzymes, including lipoproteinlipase (LPL), glycerol kinase (GK) and glycerol phosphate dehydrogenase (GPO). Triglycerides are hydrolyzed by LPL to liberate glycerol and free fatty acids. Glycerol is converted to glycerol-3-phosphate (G3P) and ADP by GK and ATP, G3P is then converted by GPO to dihydroxyacetone phosphate (DAP) and hydrogen peroxide which causes a color change.

### EXAMPLES

### Example 1

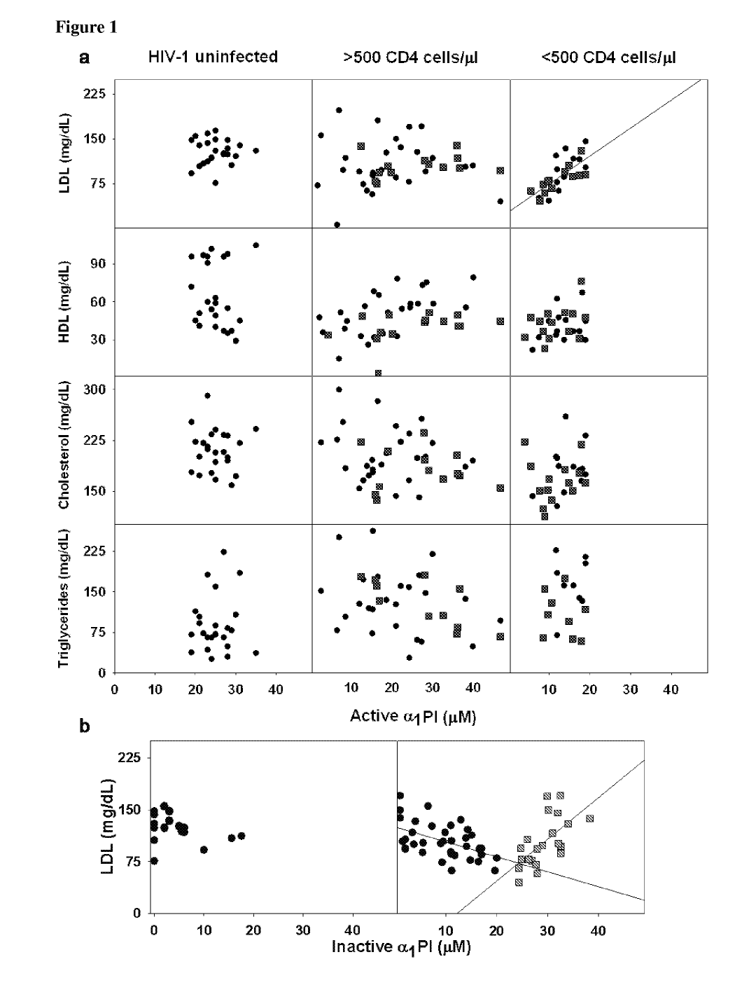

The following data demonstrate a relationship between α₁PI levels and LDL levels. In healthy individuals, the normal range of active α₁PI is 18-53 μM and 98% is in the active form ¹⁶. Inactive α₁PI arises during infection or inflammation. Active, but not inactive α₁PI, binds HLE_{CS}¹⁶ and inactive, but not active α₁PI binds LDL receptors ¹². Consistent with previous reports ¹⁴. The data presented below demonstrate that many HIV-1 patients with below normal CD4 counts exhibit below normal levels of active α₁PI (median 17 μM) and above normal levels of inactive α₁PI (median 33 μM). In patients with <500 CD4 cells/ill, higher active α₁PI was correlated with higher LDL (r²=0.44, p<0.001, n=24) whereas in HIV-1 patients with >500 CD4 (n=46) and in non-HIV-1 volunteers (n=18) LDL levels were not correlated with active α₁PI (FIG. 1a). Lipoprotein levels were not related to HLE_{CS}, CXCL12, CD184, or CD4 numbers (Table 2).

TABLE 2
Correlation between lipoprotein levels
and α₁PI, CXCL12, and lymphocyte numbers

| | Cholesterol | HDL | LDL | Triglycerides |
|---|---|---|---|---|
| activeα₁PI | 0.05 (27) | 0.00 (27) | 0.66 (24)** | 0.19 (27) |
| inactive α₁PI | 0.05 (27) | 0.00 (27) | 0.66 (24)** | 0.19 (27) |
| CXCL12 | 0.00 18) | 0.04 (16) | 0.02 (12) | 0.01 (18) |
| HLE_{CS} | 0.09 (17) | 0.00 (15) | 0.14 (11) | 0.13 (17) |
| CD4 | 0.01 (36) | 0.03 (36) | 0.01 (32) | 0.01 (38) |
| CD184 | 0.02 (18) | 0.18 (16) | 0.00 (12) | 0.00 (18) |
| CD195 | 0.00 (18) | 0.11 (16) | 0.00 (12) | 0.00 (18) |

*Values represent correlation coefficients (r) and number of observations (n). CXCL12 (pM) was measured by ELISA, and α₁PI (μM) was measured for active concentration. Absolute numbers of lymphocytes were counted by flow cytometry using whole blood analysis. Lipid levels (mg/dL) were measured by a contractor medical lab and extracted during chart review.
**p < 0.001

In contrast, inactive α₁PI correlated with LDL levels irrespective of CD4 counts, but exhibited bimodal distribution (FIG. 1b). In patients with <20 μM inactive α₁PI, lower inactive α₁PI correlated with higher LDL (r²=0.35, p<0.001, n=33) suggesting higher inactive α₁PI diminished LDL. This is consistent with reports that inactive α₁PI facilitates the binding of LDL to LDL receptors thereby decreasing free LDL ¹⁷. As expected, in these patients, lower inactive α₁PI correlated with higher active α₁PI (r²=0.24, p=0.003, n=34, not shown). On the other hand, consistent with an inflammation-induced LDL rise ¹⁸, in patients with >20 μM inactive α₁PI, higher inactive α₁PI correlated with higher LDL (r²=0.29, p<0.02, n=19). The LDL therapeutic target is 100 mg/dL and corresponded to 28 μM inactive α₁PI in this population supporting the observations that atherosclerosis is related to low active α₁PI ¹⁹ or high inactive α₁PI ¹⁸. In non-HIV-1 individuals, inactive α₁PI was 0-18 μM, (median=31.04) and as expected was below the threshold to be related to LDL levels.

### Example 2

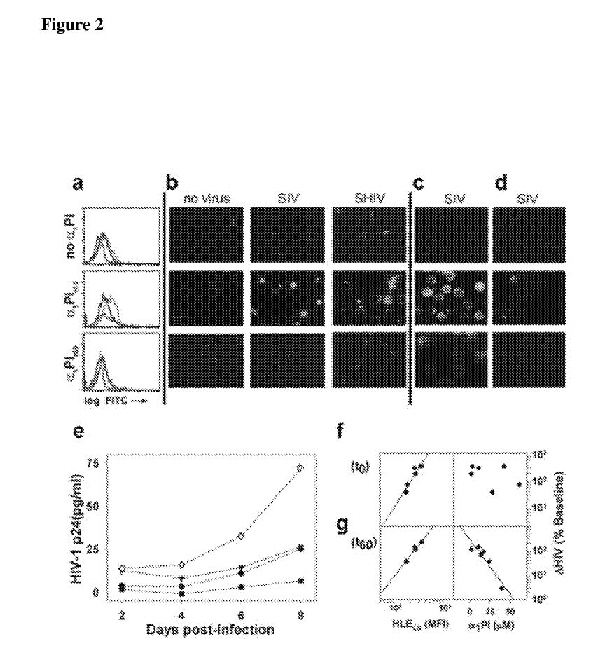

Binding of active α₁PI to HLE_{CS} produces inactive α₁PI and induces receptor polarization and cell migration ^{8,22}, activities that are time-ordered. To discriminate the effects of active α₁PI binding to HLE_{CS} versus inactive α₁PI binding to LDL receptors, cells were exposed to α₁PI for 15 min versus 60 min, and outcome was determined using HIV-1 localization. Primary monocyte-derived dendritic cells (MDC) and promonocytic U937 clone 10 cells were preconditioned with α₁PI at 37° C. to induce polarization, and subsequently exposed at 2° C. to two CD184-using non-infectious HIV-1 strains ²³. These conditions allow binding, but prevent internalization of virus. In the absence of purified α₁PI, little or no virus binding was detected (FIG. 2a-d, top panel). When virus was added to cells preconditioned with α₁PI for 15 min, considerable bound virus was detected (FIG. 2a-d, middle panel). In contrast, preconditioning cells with α₁PI for 60 min prior to the addition of virus resulted in few virus-bound cells (FIG. 2a-d, lower panel).

Infectious CD184-using HIV-1 was co-cultured with clone 10 after preconditioning cells with purified α₁PI for 0, 15, or 60 min in serum-free medium as previously described ⁸. Cells preconditioned with purified α₁PI for 15 min were productively infected with HIV-1 at levels comparable to previous reports ²⁴. In the absence of α₁PI or when cells were preconditioned for 60 min, no infectivity was detected (FIG. 2e). Long-term infectivity kinetics were not examined due to slower growth and virus yield by cells in serum-free medium ⁶.
To examine the kinetic effect of α₁PI on HIV-1 binding and infectivity of primary peripheral blood mononuclear cells (PBMC) from non-HIV-1 volunteers, cells were co-cultured with a CD195-using primary clinical isolate in the presence of 20% autologous serum with final culture concentrations of 0.8-13 μM active α₁PI/2×10⁵ cells. Cells were placed in culture with no added α₁PI or were spiked with 3 μM purified α₁PI at 0 and 60 min prior to addition of HIV-1. Change in infectivity (ΔHIV) due to the addition of α₁PI was compared using the ratio of HIV-1 produced in α₁PI-spiked versus non-spiked culture medium. It was found that higher ΔHIV correlated with higher HLE_{CS} whether the cultures were exposed to HIV-1 before or after spiking with α₁PI (FIG. 2f,g) suggesting that in vitro HIV-1 infectivity is dependent on HLE_{CS} levels. When exogenous α₁PI and HIV-1 were introduced to cells simultaneously (t₀), ΔHIV and α₁PI were not correlated (FIG. 2f); however, when cells were preconditioned with purified α₁PI for 60 min, lower ΔHIV correlated with higher α₁PI levels (FIG. 2g) suggesting α₁PI inhibits HIV-1 infectivity in vitro in a dose dependent manner when HIV-1 is introduced after prolonged α₁PI incubation. Neither CD184, nor CD195 levels were related to ΔHIV on lymphocytes or monocytic cells (Table 3).

TABLE 3
Correlation between ΔHIV, α₁PI, and blood cells

| | ΔHIV t₀ | ΔHIV t₆₀ |
|---|---|---|
| α₁PI | −0.48 | −0.95 ** |
| Mo HLE_{CS} | 0.93 ** | 0.96 ** |
| Mo CD4 | 0.76 | 0.97 ** |
| Mo CXCR4 | 0.63 | 0.76 |
| Mo CCR5 | 0.65 | 0.67 |
| Ly HLE_{CS} | −0.58 | −0.62 |
| Ly CD4 | −0.85 ** | −0.93 |
| Ly CXCR4 | 0.40 | 0.39 |
| Ly CCR5 | 0.22 | 0.24 |

* HIV-1 outcome was measured on day 6 of co-culture using PBMC from 6 volunteers and a primary isolate of CD195-using HIV-1. PBMC were spiked with α₁PI 0 min or 60 min prior to addition of HIV-1. ΔHIV is calculated as 100*(HIV spiked with α₁PI)/(HIV without spiking). Values represent Pearson correlation coefficients (r) and stars designate significance (p < 0.04). Lymphocytes (Ly) and CD14⁺ monocytic cells (Mo) were analyzed by flow cytometry using whole blood. MFI of probed receptors is depicted. α₁PI (μM) was measured as the active concentration. ΔHIV was negatively correlated with CD4⁺ lymphocytes at t₀ (r² = 0.72, p = 0.03, n = 6) and positively correlated with CD4⁺ monocytic cells at t₆₀ (r² = 0.94, p = 0.03, n = 4), consistent with the tropism of CD195-using virus.

### Example 3

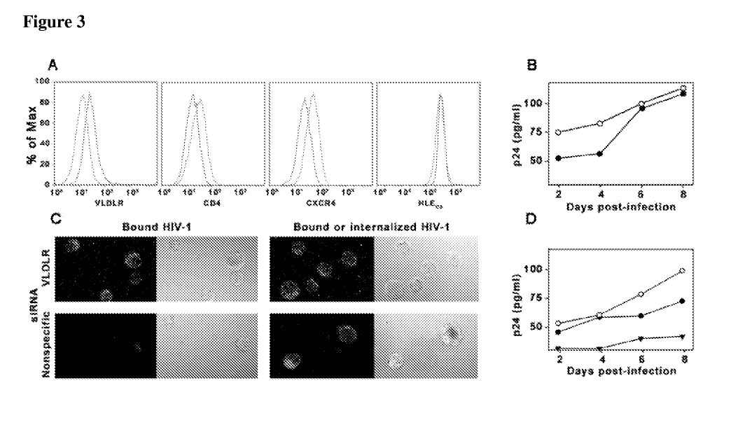

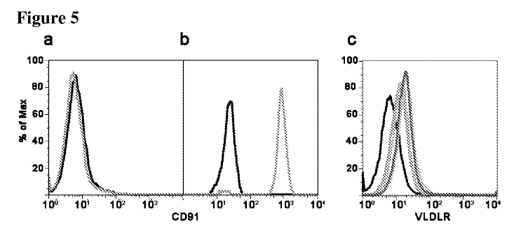

We considered that the opposing early and late influence of α₁PI on receptor polarization may involve LDL receptor-mediated endocytosis. To examine this possibility, receptors and HIV-1 binding were measured after downregulating LDL receptors. U937 clone 10 cells were transfected with LDL receptor-specific siRNA. Flow cytometric analysis determined that clone 10 cells express the VLDL receptor (VLDLR), but not LDL receptor-related protein (LRP, CD91) (FIGS. 5a, b and c). After 48 hrs, VLDLR siRNA reduced the number of VLDLR-expressing cells in a linear dose-dependent manner relative to nonspecific or LRP-specific negative control siRNA (data not shown). At optima, relative to nonspecific siRNA, VLDLR siRNA resulted in 57% VLDLR expression (FIG. 3a). As compared with negative control siRNA, VLDLR siRNA resulted in 44% and 100% increased surface expression of recycling receptors CD4 and CXCR4, respectively, and 10% decreased expression of HLE_{CS} (FIG. 3a).

VLDLR siRNA also resulted in delayed HIV-1 infectivity (FIG. 3b). However, 6 days post-infection, the rate of p24 accumulation in cells transfected with VLDLR siRNA was no different from cells transfected with negative control siRNA. These results suggest that blocking VLDLR-mediated endocytosis blocks CD4 and CXCR4 recycling without blocking HIV-1 binding. To examine this possibility, cells transfected with siRNA followed by HIV-1 incubation for 2 hr at 37° C. were examined by confocal microscopy. Cells transfected with VLDLR siRNA exhibited polarization as previously reported (Bristow et al., 2003b), and HIV-1 was detected on both non-permeabilized and permeabilized cells suggesting that HIV-1 had not internalized (FIG. 3c). In contrast, cells transfected with nonspecific siRNA exhibited polarization, but HIV-1 was only detected in permeabilized cells suggesting that HIV-1 had internalized. These results confirm that VLDLR siRNA transiently blocks endocytosis of CD4 and CXCR4 without blocking HIV-1 binding.

Unlike transitory siRNA inhibition, RAP provides continuous VLDLR blocking. Clone 10 cells were preconditioned with low endotoxin recombinant RAP for 15 min in serum-free medium prior to addition of α₁PI. Preconditioned cells were co-cultured with HIV-1 and after removal of unbound HIV-1, maintained in culture for 8 days in the presence of the same concentrations of RAP and α₁PI. Under these conditions, RAP inhibited HIV-1 infectivity 93% (FIG. 3d). In the presence of α₁PI, cells cultured with RAP were 81±10% viable at the end of the culture period. In the absence of α₁PI, cells cultured with RAP were 16±10% viable.

### Example 4

Active α₁PI, inactive α₁PI and LDL participate in a feedback regulatory pathway. Two HIV-1 patients with disease-associated α₁PI deficiency were administered therapeutic α₁PI (Table 4).

TABLE 4
Zemaira ® study population at baseline

| | | | | | | HIV |
|---|---|---|---|---|---|---|
| | | | | | | RNA |
| HIV-1 | | | HIV-1⁺ | α₁PI | CD4 | copies/ |
| Patient ^{a} | NRT/NNRT/PI ^{b} | Age | since | (μM) | cells/μl | ml |
| Alpha | Epivir/Sustiva/none | 47 | 2001 | 9 | 297 | <400 |
| Beta | Combivir/Sustiva/none | 53 | 1982 | 7 | 276 | <400 |

^{a} Patients were at different stages of disease progression and were on antiretroviral medication with adequate suppression of virus. Patients received 8-12 weekly infusions of α₁PI augmentation therapy at a dose of 120 mg/kg (Zemaira ®, lot# C405702, contributed by CSL Behring). No adverse effects of treatment were reported by any patient.
^{b} Antiretroviral medications: nucleoside reverse transcriptase inhibitor (NRT), non nucleotide reverse transcriptase inhibitor (NNRTI), aspartyl protease inhibitors lopinavir/ritonavir (PI)

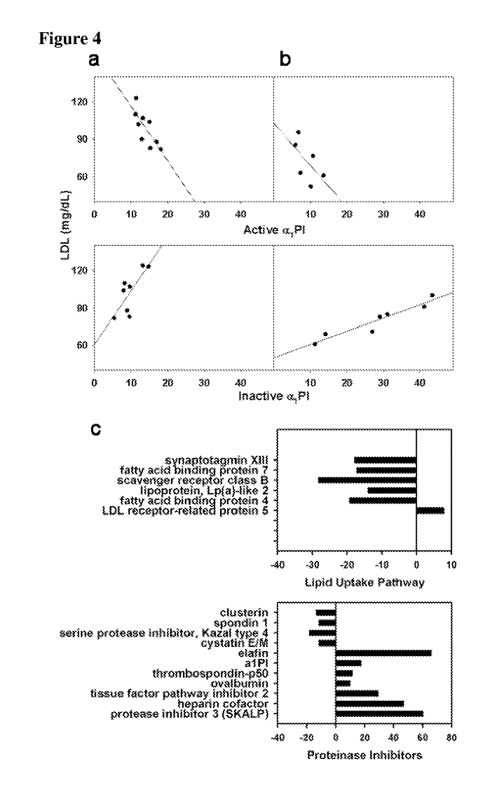

LDL levels significantly decreased as active α₁PI increased (r²=0.61, p=0.03, n=9; r²=0.42, p<0.0001, n=6) (FIG. 4a,b) and significantly increased as inactive α₁PI increased (r²=0.58, p=0.03, n=8; r²=0.90, p<0.001, n=7). There were no untoward effects of treatment. These results suggest that both active and inactive α₁PI influence LDL transport.

LDL is inflammatory and induces IL-6 ²⁰ which up-regulates α₁PI synthesis thereby increasing circulating active α₁PI. To examine the possibility that LDL and α₁PI might participate in a regulatory pathway at the cellular level, cDNA microarray analysis was performed on 14,500 functionally characterized genes using monocytic cells harvested from HIV patients receiving ritonavir, an HIV-1 protease inhibitor therapy known to elevate LDL levels ²¹. Gene expression ratios of patient to healthy cells showed that α₁PI expression was increased 18-fold, and 6 additional proteinase inhibitors were increased more than 12-fold (FIG. 4c). Of the 7 proteinase inhibitors exhibiting major up-regulation, 5 bind HLE including SKALP, ovalbumin, thrombospondin, α₁PI, and elafin; 1 binds α₁PI (heparin cofactor); 1 binds LDL (Tissue Factor Pathway Inhibitor). It was found that 4 other proteinase inhibitors were decreased in expression 12-fold or more, but none of these inhibitors are known to bind HLE, α₁PI, or lipoproteins. In contrast, 5 of 5 LDL-binding lipoproteins were decreased more than 14-fold. LDL receptor and LDL receptor-related protein 5 (LRP5) were increased 4-fold and 8-fold, respectively. This is the first demonstration that active α₁PI, inactive α₁PI, and LDL physiologically participate in a feedback regulatory pathway.

### Materials

The invention and results described herein are performed with, but not limited to, the following materials and methods

### Human Subjects.

Informed consent was obtained from all participants. Blood was collected from 24 HIV-1 seronegative, healthy adults, 12 males and 12 females and from 126 HIV-1 seropositive adults attending clinic, 38 males and 2 females at Cabrini Medical Center and 57 males and 29 females at NY Presbyterian Weill Cornell Medical Center which were measured by the respective hospital labs for complete blood count, lymphocyte phenotype, and lipids. Inclusion criteria for initiating α₁PI augmentation therapy in 3 HIV-1 patients were: i) active α₁PI below 11 μM; ii) one year history with CD4⁺ lymphocytes between 150 and 300 cells/4 iii) absence of HIV-1 disease progression; iv) adequate suppression of virus (<50 HIV RNA/ml); and v) history of compliance with antiretroviral medication. CSL Behring contributed a sufficient quantity of Zemaira® (lot# C405702) for administration of 8 weekly infusions at a dose of 120 mg/kg. Blood was collected at each session and was sent to a contractor medical laboratory for independent assessment of complete blood count, lipids, blood chemistry, lymphocyte phenotype, and HIV RNA. No adverse effects were reported by any patient.

### Animals.

Blood was collected from male and female adult macaques (Macaca mulatta) housed in the Tulane Regional Primate Research Center as previously described (Messmer et al., 2002a). Animal care operations were in compliance with the regulations detailed under the animal welfare act, and in the Guide for the Care and Use of Laboratory Animals. Before use, all animals used in this study tested negative for antibodies recognizing SIV, type D retroviruses, and simian T cell leukemia virus type 1.

### Flow Cytometric Analysis of Receptor Expression.

Surface staining on whole blood or cell suspensions were performed as previously described using ASR antibodies (BD Biosciences, San Jose, Calif.) (Bristow et al., 2008b). HLE_{CS} was detected using FITC-conjugated rabbit anti-HLE (Biodesign, Kennebunkport, Me.) or using rabbit anti-HLE (Biodesign) and negative control rabbit IgG (Chemicon, Temecula, Calif.) which had been conjugated to Alexa Fluor 647 (Molecular Probes, Eugene, Oreg.). Monoclonal anti-VLDLR (6A6, Santa Cruz Biotechnology, Santa Cruz, Calif.) and isotype control were detected using FITC-conjugated goat anti-mouse IgG.

### Cell Culture and Preconditioning.

U937 subclones have been previously characterized (Franzoso et al., 1994; Bristow et al., 2008a). Immature and mature monocyte-derived dendritic cells (MDC) were generated from human or macaque peripheral blood mononuclear cells (PBMC) and phenotype was confirmed by flow cytometry for each experiment as previously described (Messmer et al., 2002a). To stimulate receptor polarization, cells were preconditioned with active-site standardized α₁PI (Sigma, cat# A9024 or Zemaira®, CSL Behring) or negative control buffer for various time points at 37° C. In some cases, U937 clone 10 cells in serum-free medium (1×10⁶) were incubated with or without 3 nmol low endotoxin recombinant RAP (Molecular Innovations, Southfield, Mich.) or negative control buffer for 15 min at 37° C. prior to addition of α₁PI.

VLDLR Targeted siRNA.

U937 clone 10 cells in RPMI 1640 containing 10% FBS were transfected with LRP-1 siRNA (Ambion, Austin, Tex., ID#106762; SEQ ID NO: 2), VLDLR siRNA (Ambion, ID#111310; SEQ ID NO: 1), or negative control siRNA (Ambion, ID#1; SEQ ID NO: 3). Successful delivery of siRNA was achieved for 2.5×10⁵ cells suspended in 250 μl culture media containing 0.15-30 nmol individual siRNA by passage through a syringe fitted with a 25-gauge needle as previously described (Bristow et al., 2003b). Minimal VLDLR expression relative to negative control siRNA using U937 clone 10 cells was achieved 48 hr after transfecting 2 nmol siRNA/1×10⁶ cells. Cells were measured for cell viability and for expression of CD91 (LRP), VLDLR, CD4, CXCR4, and HLE_{CS} by flow cytometric analysis.

### HIV-1 Infectivity of U937 Cells.

Without transferring cells from Eppendorf tubes, cells were co-cultured with HIV-1 NL4-3 (TCID₅₀=10^{-5.17}, Advanced Biotechnologies, Inc., Columbia, Md.) for 2 h at 37° C. Cells were washed and resuspended in 0.7 ml fresh medium containing the α₁PI or RAP at concentrations matching preconditioning concentrations. Negative control cells were incubated with α₁PI in the presence of 0.214 T-20 fusion inhibitor (AIDS Research and Reference Reagent Program, NIH). Culture supernatant (100 μl) was collected without replacement of fresh media every other day for 8 days and stored at −80° C. for analysis of p24 antigen (ZeptoMetrix Corp., Buffalo, N.Y.). Cell counts and viability were determined on the 8^{th} day post-infection and in all cases were >85%.

### Inactivated Virus Binding.

AT-2 chemically-inactivated SIV or SHIV preparations, non-infectious virus with conformationally and functionally intact envelope glycoproteins, were provided by the AIDS Vaccine Program (SAIC-Frederick, National Cancer Institute at Frederick, Frederick, Md., USA) (Rossio et al., 1998; Frank et al., 2002). The SIVmneE11S virus was produced from a chronically infected subclone of HuT-78 designated HuT-78c1.E11S. The SHIV89.6 virus was produced from the CEM X 174 (T1) cell line. Virus content of purified concentrated preparations was determined with an antigen capture immunoassay for the SIV gag p27 or HIV p24 (AIDS Vaccine Program).

MDC or U937 clone 10 were cultured in AIM V(R) serum-free medium (Gibco/Invitrogen) for 24 h and resuspended at 1×10⁷/ml AIM V(R) prior to preconditioning. To prevent virus internalization, all reagents were allowed to equilibrate to 2° C. prior to virus pulsing of preconditioned cells. After pulsing cells with virus (30 ng p27 or p24/10⁶ cells) for 2 h at 2° C. cells were washed using ice cold Dulbecco's PBS for flow cytometric analysis or for adherence to Alcian blue-coated slides respectively (Frank et al., 2002).

### Viral Envelope-Staining

Cells attached to Alcian blue slides for microscopy or maintained in suspension for flow cytometry were fixed and stained as previously described (Frank et al., 2002). Inactivated virus was detected using biotinylated tetravalent human CD4-IgG₂ provided by Progenics Pharmaceutical Inc. (Tarrytown, N.Y., USA). Live virus was detected using dodecameric human CD4-IgG₁ (Arthos et al., 2002) which was kindly provided by the Laboratory of Immunoregulation, NIAID, NIH. Both reagents specifically recognize conformationally intact HIV-1/SIV envelope gp120. Biotinylated CD4-IgG₂ was detected using HRP-conjugated streptavidin (NEN Life Science Products), and CD4-IgG₁ was detected using HRP-conjugated Rb anti-human IgG (Sigma). CD4-IgG-labeled cells were coupled to FITC using the Tyramide signal amplification system (NEN Life Science Products, Boston, Mass., USA) as previously described (Frank et al., 2002). Cells stained on slides were permeabilized using 0.05% saponin during the blocking step and further stained with the nuclear staining dye, 4′,6-diamidino-2-phenylindole (DAPI), mounted, and examined by epifluorescence microscopy using an Olympus AX70 or by confocal microscopy using an Olympus FluoView.

### Statistical Analysis.

Multiple linear regression and computer generated curve fitting were performed using SigmaPlot. Correlation coefficients were determined by Spearman Rank Order. Measurements are presented as mean±standard deviation unless stated otherwise.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### Virus Binding.

AT-2 chemically-inactivated SIV or SHIV preparations, non-infectious virus with conformationally and functionally intact envelope glycoproteins, were provided by the AIDS Vaccine Program (SAIC-Frederick, Frederick, Md.) ^{23,29}. After pulsing cells with virus (30 ng p27 or p24/10⁶ cells) for 2 h at 2° C., cells were attached to Alcian blue slides for microscopy or maintained in suspension for flow cytometry ²³. Inactivated virus was detected using biotinylated tetravalent human CD4-IgG₂ (Progenics Pharmaceutical Inc. Tarrytown, N.Y., USA). NL4-3 live virus was detected using dodecameric human CD4-IgG₁ ³⁰ provided by the Laboratory of Immunoregulation, NIAID, NIH. Biotinylated CD4-IgG₂ was detected using HRP-conjugated streptavidin (NEN Life Science Products), and CD4-IgG₁ was detected using HRP-conjugated Rb anti-human IgG (Sigma). CD4-IgG-labeled cells were coupled to FITC using the Tyramide signal amplification system (NEN Life Science Products, Boston, Mass., USA).

### Gene Expression in Monocytic Cells.

Total RNA (50 ng) was extracted from non-adherent PBMC from 1 non-HIV-1 donor and 2 HIV-1 patients on protease inhibitor therapy (ritonavir), purified using RNeasy mini kit (Qiagen, Chatsworth, Calif.), and amplified, yielding an average of 7 μg of single-stranded cDNA which was fragmented and labeled with biotin using the Ovation Biotin system (NuGEN Technologies, Inc., San Carlos, Calif.). Expression patterns of 18,400 genes, 14,500 functionally characterized genes and 3,900 expressed sequence tag clusters, were examined using GeneChip U133A2.0 arrays (Affymetrix, Santa Clara, Calif.). Data were analyzed by large-scale microarrays involving two independent primary culture preparations and DNA microarray runs.

### REFERENCES

Ali, H., Tomhave, E. D., Richardson, R. M., Haribabu, B., and Snyderman, R. (1996). Thrombin primes responsiveness of selective chemoattractant receptors at a site distal to G protein activation. J. Biol. Chem. 271, 3200-3206.
Andrade, N., Komnenovic, V., Blake, S. M., Jossin, Y., Howell, B., Goffinet, A., Schneider, W. J., and Nimpf, J. (2007). ApoER2/VLDL receptor and Dab1 in the rostral migratory stream function in postnatal neuronal migration independently of Reelin. PNAS. 104, 8508-8513.
Arthos, J., Cicala, C., Steenbeke, R. D., Chun, T.-W., Dela Cruz, C., Hanback, D. B., Khazanie, P., Nam, D., Schuck, P., Selig, S. M., Van Ryk, D., Chaikin, M. A., and Fauci, A. S. (2002). Biochemical and biological characterization of a dodecameric CD4-Ig fusion protein. J. Biol. Chem. 277, 11456-11464.
Banda, M. J., Rice, A. G., Griffin, G. L., and Senior, R. M. (1988). α1-proteinase inhibitor is a neutrophil chemoattractant after proteolytic inactivation by macrophage elastase. J. Biol. Chem. 263, 4481-4484.
Bangalore, N. and Travis, J. (1994). Comparison of properties of membrane bound versus soluble forms of human leukocytic elastase and cathepsin G. Biol. Chem. Hoppe-Seyler. 375, 659-666.
Bartosch, B., Vitelli, A., Granier, C., Goujon, C., Dubuisson, J., Pascale, S., Scarselli, E., Cortese, R., Nicosia, A., and Cosset, F. L. (2003). Cell Entry of Hepatitis C Virus Requires a Set of Co-receptors That Include the CD81 Tetraspanin and the SR-B1 Scavenger Receptor. J. Biol. Chem. 278, 41624-41630.
Berninger, R. W. (1985). Alpha 1-antitrypsin. J. Med. 16, 23-99.
Brantly, M. L., Wittes, J. T., Vogelmeier, C. F., Hubbard, R. C., Fells, G. A., and Crystal, R. G. (1991). Use of a highly purified alpha 1-antitrypsin standard to establish ranges for the common normal and deficient alpha 1-antitrypsin phenotypes. Chest 100, 703-708.
Bristow, C. L., Cortes, J., Mukhtarzad, R., Trucy, M., Franklin, A., Romberg, V., and Winston, W. (2009). α1Antitrypsin therapy increases CD4⁺ lymphocytes to normal values in HIV-1 patients. In Soluble Factors Mediating Innate Immune Responses to HIV Infection, M. Alfano, ed. Bentham Science Publishers, In Press).
Bristow, C. L., di Meo, F., and Arnold, R. R. (1998). Specific activity of α1proteinase inhibitor and α2macroglobulin in human serum: Application to insulin-dependent diabetes mellitus. Clin. Immunol. Immunopathol. 89, 247-259.
Bristow, C. L., Fiscus, S. A., Flood, P. M., and Arnold, R. R. (1995). Inhibition of HIV-1 by modification of a host membrane protease. Int. Immunol. 7, 239-249.
Bristow, C. L. and Flood, P. M. (1993). T cell antigen receptor immune complexes demonstrating biologic and proteolytic activity. Int Immunol 5, 79-88.
Bristow, C. L., Mercatante, D. R., and Kole, R. (2003b). HIV-1 preferentially binds receptors co-patched with cell surface elastase. Blood 102, 4479-4486.
Bristow, C. L., Mercatante, D. R., and Kole, R. (2003a). HIV-1 preferentially binds receptors co-patched with cell surface elastase. Blood 102, 4479-4486.
Bristow, C. L., Wolkowicz, R., Trucy, M., Franklin, A., Di Meo, F., Kozlowski, M. T., Winston, R., and Arnold, R. R. (2008b). NF-{kappa}B Signaling, Elastase Localization, and Phagocytosis Differ in HIV-1 Permissive and Nonpermissive U937 Clones. The Journal of Immunology 180, 492-499.
Bristow, C. L., Wolkowicz, R., Trucy, M., Franklin, A., Di Meo, F., Kozlowski, M. T., Winston, R., and Arnold, R. R. (2008d). NF-{kappa}B Signaling, Elastase Localization, and Phagocytosis Differ in HIV-1 Permissive and Nonpermissive U937 Clones. The Journal of Immunology 180, 492-499.
Bristow, C. L., Wolkowicz, R., Trucy, M., Franklin, A., Di Meo, F., Kozlowski, M. T., Winston, R., and Arnold, R. R. (2008a). NF-{kappa}B Signaling, Elastase Localization, and Phagocytosis Differ in HIV-1 Permissive and Nonpermissive U937 Clones. The Journal of Immunology 180, 492-499.
Bristow, C. L., Wolkowicz, R., Trucy, M., Franklin, A., Di Meo, F., Kozlowski, M. T., Winston, R., and Arnold, R. R. (2008c). NF-{kappa}B Signaling, Elastase Localization, and Phagocytosis Differ in HIV-1 Permissive and Nonpermissive U937 Clones. The Journal of Immunology 180, 492-499.
Bristow, C. L., Wolkowicz, R., Trucy, M., Franklin, A., Di Meo, F., Kozlowski, M. T., Winston, R., and Arnold, R. R. (2008e). NF-κB Signaling, Elastase Localization, and Phagocytosis Differ in HIV-1 Permissive and Nonpermissive U937 Clones. J. Immunol. 180, 492-499.
Cao, C., Lawrence, D. A., Li, Y., Von Amin, C. A., Herz, J., Su, E. J., Makarova, A., Hyman, B. T., Strickland, D. K., and Zhang, L. (2006). Endocytic receptor LRP together with tPA and PAI-1 coordinates Mac-1-dependent macrophage migration. EMBO J. 25, 1860-1870.
Courtney, M., Buchwalder, A., Tessier, L.-H. J. M., Benavente, A., Balland, A., Kohli, V., Lathe, R., Tolstoshev, P., and Lecocq, J. P. (1984). High-level production of biologically active human α1antitrypsin in Escherichia coli. Proc Natl Acad Sci; USA 81, 669-673.
Cselenyi, C. S., Jernigan, K. K., Tahinci, E., Thorne, C. A., Lee, L. A., and Lee, E. (2008). LRP6 transduces a canonical Wnt signal independently of Axin degradation by inhibiting GSK3's phosphorylation of +¦-catenin. PNAS. 105, 8032-8037.
Current Protocols in Molecular Biology (2002). Greene Publishing Associates and Wiley-Intersciences, New York).
Czekay, R. P., Orlando, R. A., Woodward, L., Lundstrom, M., and Farquhar, M. G. (1997). Endocytic trafficking of megalin/RAP complexes: dissociation of the complexes in late endosomes. Molecular Biology of the Cell 8, 517-532.
Dichtl, W., Moraga, F., Ares, M. P. S., Crisby, M., Nilsson, J., Lindgren, S., and Janciauskiene, S. (2000). The Carboxyl-Terminal Fragment of [alpha]1-Antitrypsin Is Present in Atherosclerotic Plaques and Regulates Inflammatory Transcription Factors in Primary Human Monocytes. Molecular Cell Biology Research Communications 4, 50-61.
Elliott, P. R., Pei, X. Y., Dafforn, T. R., and Lomas, D. A. (2000). Topography of a 2.0 A structure of alpha1-antitrypsin reveals targets for rational drug design to prevent conformational disease [In Process Citation]. Protein Sci 9, 1274-1281.
Fields, G. B. and Noble, R. L. (1990). Solid phase peptide synthesis utilizing 9-fluorenylmethoxycarbonyl amino acids. Int J Pept Protein Res. 35, 161-214.
Frank, I., Piatak, M. J., Stoessel, H., Romani, N., Bonnyay, D., Lifson, J. D., and Pope, M. (2002). Infectious and whole inactivated simian immunodeficiency viruses interact similarly with primate dendritic cells (DCs): differential intracellular fate of virions in mature and immature DCs. J. Virol. 76, 2936-2951.
Franzoso, G., Biswas, P., Poli, G., Carlson, L. M., Brown, K. D., Tomita-Yamaguchi, M., Fauci, A. S., and Siebenlist, U. K. (1994). A family of serine proteases expressed exclusively in myelo-monocytic cells specifically processes the nuclear factor-kappa B subunit p65 in vitro and may impair human immunodeficiency virus replication in these cells. J. Exp. Med. 94, 1445-1456.
Goselink, H. M., van Damme, J., Hiemstra, P. S., Wuyts, A., Stolk, J., Fibbe, W. E., Willemze, R., and Falkenburg, J. H. (1996). Colony growth of human hematopoietic progenitor cells in the absence of serum is supported by a proteinase inhibitor identified as antileukoproteinase. J. Exp. Med. 184, 1305-1312.
Janciauskiene, S. and Lindgren, S. (1999). Effects of fibrillar C-terminal fragment of cleaved alpha1-antitrypsin on cholesterol homeostasis in HepG2 cells. Hepatology 29, 434-442.
Janciauskiene, S., Wright, H. T., and Lindgren, S. (1999a). Atherogenic properties of human monocytes induced by the carboxyl terminal proteolytic fragment of alpha-1-antitrypsin. Atherosclerosis 147, 263-275.
Janciauskiene, S., Wright, H. T., and Lindgren, S. (1999b). Atherogenic properties of human monocytes induced by the carboxyl terminal proteolytic fragment of alpha-1-antitrypsin. Atherosclerosis 147, 263-275.
Janciauskiene, S., Moraga, F., and Lindgren, S. (2001). C-terminal fragment of [alpha]1-antitrypsin activates human monocytes to a pro-inflammatory state through interactions with the CD36 scavenger receptor and LDL receptor. Atherosclerosis 158, 41-51.
Jean, F., Stella, K., Thomas, L., Lui, G., Xiang, Y., and Reason, A. J. (1998). α1-antitrypsin Portland, a bioengineered serpin hihly selective for furin: Application as an antipathogenic agent. Proc Natl Acad Sci; USA 95, 7293-7298.
Jeppsson, J. O., Lilja, H., and Johansson, M. (1985). Isolation and characterization of two minor fractions of alpha 1-antitrypsin by high-performance liquid chromatographic chromatofocusing. J. Chromatogr. 327, 173-177.
Jo slin, G., Griffin, G. L., August, A. M., Adams, S Fallon, R. J., Senior, R. M., and Perlmutter, D. H. (1992). The serpin-enzyme complex (SEC) receptor mediates the neutrophil chemotactic effect of α-₁antitrypsin-elastase complexes and amyloid-β peptide. J. Clin. Invest. 90, 1150-1154.
Kindzelskii, A. L. and Petty, H. R. (2003). Intracellular Calcium Waves Accompany Neutrophil Polarization, Formylmethionylleucylphenylalanine Stimulation, and Phagocytosis: A High Speed Microscopy Study. J. Immunol. 170, 64-72.
Kounnas, M. Z., Church, F. C., Argraves, W. S., and Strickland, D. K. (1996). Cellular internalization and degradation of antithrombin III-thrombin, heparin cofactor II-thrombin, and alpha 1-antitrypsin-trypsin complexes is mediated by the low density lipoprotein receptor-related protein. J. Biol. Chem. 271, 6523-6529.
Kushner, I. (1982). The phenomenon of the acute phase response. Ann. N. Y. Acad. Sci. 389, 39-47.
Lapidot, T. and Petit, I. (2002). Current understanding of stem cell mobilization: The roles of chemokines, proteolytic enzymes, adhesion molecules, cytokines, and stromal cells. Exp. Hematol. 30, 973-981.
Marlovits, T. C., Abrahamsberg, C., and Blaas, D. (1998). Very-Low-Density Lipoprotein Receptor Fragment Shed from HeLa Cells Inhibits Human Rhinovirus Infection. J. Virol. 72, 10246-10250.
Mellet, P., Boudier, C., Mely, Y., and Bieth, J. G. (1998). Stopped Flow Fluorescence Energy Transfer Measurement of the Rate Constants Describing the Reversible Formation and the Irreversible Rearrangement of the Elastase-alpha 1-Proteinase Inhibitor Complex. Journal of Biological Chemistry 273, 9119-9123.
Messmer, D., Jacque, J. M., Santisteban, C., Bristow, C. L., Han, S. Y., Villamide-Herrera, L., Mehlhop, E. R., Marx, P. A., Steinman, R. M., Gettie, A., and Pope, M. (2002a). Endogenously expressed nef uncouples cytokine and chemokine production from membrane phenotypic maturation in dendritic cells. J. Immunol. 169, 4172-4182.
Messmer, D., Jacque, J.-M., Santisteban, C., Bristow, C. L., Han, S.-Y., Villamide-Herrera, L., Mehlhop, E. R., Marx, P. A., Steinman, R. M., Gettie, A., and Pope, M. (2002b). Endogenously expressed nef uncouples cytokine and chemokine production from membrane phenotypic maturation in dendritic cells. J. Immunol. 169, 4172-4182.
Misra, U. K. and Pizzo, S. V. (2001). Receptor-Associated Protein Binding Blocks Ubiquitinylation of the Low Density Lipoprotein Receptor-Related Protein. Arch. Biochem. Biophys. 396, 106-110.
Nukiwa, T., Satoh, K., Brantly, M. L., Ogushi, F., Fells, G. A., Courtney, M., and Crystal, R. G. (1986). Identification of a second mutation in the protein-coding sequence of the Z type alpha 1-antitrypsin gene. J. Biol. Chem. 261, 15989-15994.
OMIM. Online Mendelian Inheritance in Man, OMIM™. McKusick-Nathans Institute for Genetic Medicine, Johns Hopkins University (Baltimore, Md.) and National Center for Biotechnology Information, National Library of Medicine (Bethesda, Md.). 2000. Ref Type: Data File
Parfrey, H., Mahadeva, R., Ravenhill, N. A., Zhou, A., Dafforn, T. R., Foreman, R. C., and Lomas, D. A. (2003). Targeting a surface cavity of α_{1-antitrypsin to prevent conformational disease}. J. Biol. Chem. 278, 33060-33066.
Perkins, S. J., Smith, K. F., Nealis, A. S., Haris, P. I., Chapman, D., Bauer, C. J., and Harrison, R. A. (1992). Secondary structure changes stabilize the reactive-centre cleaved form of SERPINs. J. Mol. Biol. 228, 1235-1254.
Poller, W., Willnow, T. E., Hilpert, J., and Herz, J. (1995). Differential recognition of alpha 1-antitrypsin-elastase and alpha 1-antichymotrypsin-cathepsin-G complexes by the low density lipoprotein receptor-related protein. J. Biol. Chem. 270, 2841-2845.
Robert, J., Ramanayake, T., Maniero, G. D., Morales, H., and Chida, A. S. (2008). Phylogenetic Conservation of Glycoprotein 96 Ability to Interact with CD91 and Facilitate Antigen Cross-Presentation. The Journal of Immunology 180, 3176-3182.
Rodenburg, K. W., Kjoller, L., Petersen, H. H., and Andreasen, P. A. (1998). Binding of urokinase-type plasminogen activator-plasminogen activator inhibitor-1 complex to the endocytosis receptors alpha2-macroglobulin receptor/low-density lipoprotein receptor-related protein and very-low-density lipoprotein receptor involves basic residues in the inhibitor. Biochemical Journal 329, 55-63.
Rossio, J. L., Esser, M. T., Suryanarayana, K., Schneider, D. K., Bess, J. W., Jr., Vasquez, G. M., Wiltrout, T. A., Chertova, E., Grimes, M. K., Sattentau, Q., Arthur, L. O., Henderson, L. E., and Lifson, J. D. (1998). Inactivation of human immunodeficiency virus type 1 infectivity with preservation of conformational and functional integrity of virion surface proteins. J. Virol. 72, 7992-8001.
Shulman, Z., Shinder, V., Klein, E., Grabovsky, V., Yeger, O., Geron, E., Montresor, A., Bolomini-Vittori, M., Feigelson, S. W., Kirchhausen, T., Laudanna, C., Shakhar, G., and Alon, R. (2009). Lymphocyte Crawling and Transendothelial Migration Require Chemokine Triggering of High-Affinity LFA-1 Integrin. Immunity 30, 384-396.
Sifers, R. N., Brashears-Macatee, S., Kidd, V. J., Muensch, H., and Woo, S. L. (1988). A frameshift mutation results in a truncated alpha 1-antitrypsin that is retained within the rough endoplasmic reticulum. Journal of Biological Chemistry 263, 7330-7335.
Terashima, M., Murai, T., Kawamura, M., Nakanishi, S., Stoltz, T., Chen, L., Drohan, W., Rodriguez, R. L., and Katoh, S. (1999). Production of functional human α1-antitrypsin by plant cell culture. Appl Microbiol Biotechnol 52, 516-523.
Weaver, A. M., Hussaini, I. M., Mazar, A., Henkin, J., and Gonias, S. L. (1997). Embryonic Fibroblasts That Are Genetically Deficient in Low Density Lipoprotein Receptor-related Protein Demonstrate Increased Activity of the Urokinase Receptor System and Accelerated Migration on Vitronectin. Journal of Biological Chemistry 272, 14372-14379.
Wright, S. D. and Meyer, B. C. (1986). Phorbol esters cause sequential activation and deactivation of complement receptors on polymorphonuclear leukocytes. J. Immunol. 136, 1759-1764.
Zhadina, M., McClure, M. O., Johnson, M. C., and Bieniasz, P. D. (2007). Ubiquitin-dependent virus particle budding without viral protein ubiquitination. PNAS. 104, 20031-20036.

### REFERENCES

1. Mu, H. & Hoy, C.-E. The digestion of dietary triacylglycerols. Prog. Lipid Res. 43, 105-133 (2004).
2. Spijkers, P. P. E. M. et al. LDL-receptor-related protein regulates {beta}2-integrin-mediated leukocyte adhesion. Blood 105, 170-177 (2005).
3. Mantuano, E., Jo, M., Gonias, S. L. & Campana, W. M. Low Density Lipoprotein Receptor-related Protein (LRP1) Regulates Rac1 and RhoA Reciprocally to Control Schwann Cell Adhesion and Migration. J. Biol. Chem. 285, 14259-14266 (2010).
4. Strickland, D. K., Gonias, S. L. & Argraves, W. S. Diverse roles for the LDL receptor family. Trends Endocrinol. Metab. 13, 66-74 (2002).
5. Bangalore, N. & Travis, J. Comparison of properties of membrane bound versus soluble forms of human leukocytic elastase and cathepsin G. Biol. Chem. Hoppe-Seyler. 375, 659-666 (1994).
6. Bristow, C. L. et al. NF-κB Signaling, Elastase Localization, and Phagocytosis Differ in HIV-1 Permissive and Nonpermissive U937 Clones. J. Immunol. 180, 492-499 (2008).
7. Cao, C. et al. Endocytic receptor LRP together with tPA and PAI-1 coordinates Mac-1-dependent macrophage migration. EMBO J. 25, 1860-1870 (2006).
8. Bristow, C. L., Mercatante, D. R. & Kole, R. HIV-1 preferentially binds receptors co-patched with cell surface elastase. Blood 102, 4479-4486 (2003).
9. Clark, A. J. The Mode of Action of Drugs on Cells. E. Arnold and Co., London (1933).
10. Shulman, Z. et al. Lymphocyte Crawling and Transendothelial Migration Require Chemokine Triggering of High-Affinity LFA-1 Integrin. Immunity 30, 384-396 (2009).
11. Kounnas, M. Z., Church, F. C., Argraves, W. S. & Strickland, D. K. Cellular internalization and degradation of antithrombin III-thrombin, heparin cofactor II-thrombin, and alpha 1-antitrypsin-trypsin complexes is mediated by the low density lipoprotein receptor-related protein. J. Biol. Chem. 271, 6523-6529 (1996).
12. Janciauskiene, S., Moraga, F. & Lindgren, S. C-terminal fragment of [alpha] 1-antitrypsin activates human monocytes to a pro-inflammatory state through interactions with the CD36 scavenger receptor and LDL receptor. Atherosclerosis 158, 41-51 (2001).
13. Weaver, A. M., Lysiak, J. J. & Gonias, S. L. LDL receptor family-dependent and -independent pathways for the internalization and digestion of lipoprotein lipase-associated beta-VLDL by rat vascular smooth muscle cells. J. Lipid Res. 38, 1841-1850 (1997).
14. Bristow, C. L., Patel, H. & Arnold, R. R. Self antigen prognostic for human immunodeficiency virus disease progression. Clin Diagn. Lab. Immunol. 8, 937-942 (2001).
15. Bristow, C. L. et al. Soluble Factors Mediating Innate Immune Responses to HIV Infection. Alfano, M. (ed.), pp. 102-110 (Bentham Science Publishers, 2010).
16. Bristow, C. L., di Meo, F. & Arnold, R. R. Specific activity of α1proteinase inhibitor and α2macroglobulin in human serum: Application to insulin-dependent diabetes mellitus. Clin. Immunol. Immunopathol. 89, 247-259 (1998).
17. Janciauskiene, S., Lindgren, S. & Wright, H. T. The C-terminal peptide of α-1-antitrypsin increases low density lipoprotein binding in HepG2 cells. Eur. J. Biochem. 254, 460-467 (1998).
18. Hansson, G. Inflammatory mechanisms in atherosclerosis. J. Thromb. Haemost. 7, 328-331 (2009).
19. Talmud, P. J. et al. Progression of Atherosclerosis Is Associated With Variation in the {alpha}1-Antitrypsin Gene. Arterioscler Thromb Vasc Biol 23, 644-649 (2003).
20. Benitez, S., Bancells, C., Ordonez-Llanos, J. & Sanchez-Quesada, J. L. Pro-inflammatory action of LDL(−) on mononuclear cells is counteracted by increased IL10 production. Biochim Biophys Acta 1771, 613-622 (2007).
21. Purnell, J. Q. et al. Effect of ritonavir on lipids and post-heparin lipase activities in normal subjects. AIDS 14, 51-57 (2000).
22. Stockley, R. A., Shaw, J., Afford, S. C., Morrison, H. M. & Burnett, D. Effect of alpha-1-proteinase inhibitor on neutrophil chemotaxis. Am. J. Respir. Cell Mol. Biol. 2, 163-170 (1990).
23. Frank, I. et al. Infectious and whole inactivated simian immunodeficiency viruses interact similarly with primate dendritic cells (DCs): differential intracellular fate of virions in mature and immature DCs. J. Virol. 76, 2936-2951 (2002).
24. Wen, W., Chen, S., Cao, Y., Zhu, Y. & Yamamoto, Y. HIV-1 infection initiates changes in the expression of a wide array of genes in U937 promonocytes and HUT78 T cells. Virus Res. 113, 26-35 (2005).
25. Bristow, C. L., Fiscus, S. A., Flood, P. M. & Arnold, R. R. Inhibition of HIV-1 by modification of a host membrane protease. Int. Immunol. 7, 239-249 (1995).
26. Shapiro, L., Pott, G. B. & Ralston, A. H. Alpha-1-antitrypsin inhibits human immunodeficiency virus type 1. FASEB J. 15, 115-122 (2001).
27. Munch, J. et al. Discovery and Optimization of a Natural HIV-1 Entry Inhibitor Targeting the gp41 Fusion Peptide. Cell 129, 263-275 (2007).
28. Hussaini I M et al. Stable antisense RNA expression neutralizes the activity of low-density lipoprotein receptor-related protein and promotes urokinase accumulation in the medium of an astrocytic tumor cell line. Antisense Nucleic Acid Drug Dev. 9, 183-190 (1999).
29. Rossio, J. L. et al. Inactivation of human immunodeficiency virus type 1 infectivity with preservation of conformational and functional integrity of virion surface proteins. J. Virol. 72, 7992-8001 (1998).
30. Arthos, J. et al. Biochemical and biological characterization of a dodecameric CD4-Ig fusion protein. J. Biol. Chem. 277, 11456-11464 (2002).

What is claimed is:

1. A method of decreasing low density lipoprotein (LDL) levels in a subject comprising:
administering to the subject α1PI or at least one peptide derived from α1PI; thereby decreasing the levels of LDL in the subject.

2. A method of modulating the distribution of LDL levels, HDL levels, cholesterol levels, triglyceride levels and other lipids derived from LDL, HDL, cholesterol, and triglycerides in a subject comprising:
administering to the subject α1PI or at least one peptide derived from α1PI; thereby modulating the distribution of LDL levels, HDL levels, cholesterol levels, triglyceride levels and other lipids derived from LDL, HDL, cholesterol, and triglycerides in the subject.

3. The method of claim 1 or 2, wherein the subject is suffering from a disease or disorder selected from the group consisting of: heart disease, atherosclerosis, hypertension, HIV infection, viral infection, bacterial infection, leukemia, Alzheimer's Disease, a solid tumor, or autoimmune disease.

4. The method of claim 3, wherein the subject is suffering from HIV-1.

5. The method of claim 1 or 2, wherein the number of CD4 T cells in the subject is less than 500 CD4 cells/μl.

6. The method of claim 1 or claim 2, wherein the subject is receiving proteinase inhibitor therapy.

7. The method of claim 1 or claim 2, wherein α1PI or said at least one peptide derived from α1PI decreases LDL levels by promoting LDL endocytosis.

8. The method of claim 1 or claim 2, wherein α1PI or said at least one peptide derived from α1PI decreases LDL levels by promoting LDL transport.

9. The method of claim 1 or claim 2, wherein the LDL receptor is very low density LDL (VLDL) receptor.

10. The method of claim 1 or claim 2, wherein the subject is a human or a non-human animal.

11. The method of claim 1 or claim 2, further comprising administering an LDL inhibitor.

12. The method of claim 1 or 2, wherein the LDL inhibitor is a nucleic acid inhibitor, a small molecule inhibitor, a peptide or a peptide mimetic.

13. The method of claim 12, wherein the nucleic acid inhibitor is a siRNA.

14. The method of claim 12, wherein the peptide or peptide mimetic comprises LDL Receptor Associated Protein.

15. A method of treating a subject with a disease associated with an increase in the level of LDL, comprising:
identifying a subject in need of treatment;
administering to said subject α1PI or at least one peptide derived from α1PI;
determining the level of LDL in said subject;
wherein, following said administration, there is a decrease in the level of LDL in said subject, thereby treating said disease.

16. A method of treating a subject with a disease associated with an increase in the level of LDL, comprising;
administering to said subject α1PI or at least one peptide derived from α1PI identified as capable of decreasing the level of LDL in said subject;
determining the level of LDL in said subject;
wherein following said administration, there is a decrease in the level of LDL in said subject thereby treating said disease.

17. A method of treating a subject with a disease, comprising;
administering to said subject α1PI or at least one peptide derived from al PI identified as capable of decreasing the level of LDL in said subject wherein following said administration, there is a decrease in the level of LDL in said subject thereby treating said disease.

18. A method of monitoring the treatment of a subject diagnosed with a disease associated with an increase in the level of LDL levels comprising:
administering to said subject α1PI or at least one peptide derived from al PI; and
comparing the level of LDL of said subject before and after administration of said α1PI or at least one peptide derived from α1PI.

19. The method of claim 18, wherein following administration of said α1PI or at least one peptide derived from α1PI there is a decrease in the level of LDL in said subject thereby indicating treatment.

20. A method of monitoring the treatment of a subject diagnosed with a disease associated with an increase in the level of LDL comprising:
determining the level of LDL in said subject;
administering to said subject α1PI or at least one peptide derived from al PI; and
comparing the level of LDL of said subject with the level of LDL of a control subject that is not diagnosed with said disease.

21. The method of claim 20, wherein following administration of α1PI or at least one peptide derived from α1PI there is a decrease in the level of LDL of said subject diagnosed with said disease as compared to said control subject, thereby indicating treatment.

22. A method of treating a subject with a disease associated with an increase in the level of LDL comprising:
administering to said subject α1PI or at least one peptide derived from α1PI; and
determining the level of LDL;
wherein following said administration there is a decrease in the level of LDL thereby treating said disease.

23. A method of decreasing the level of LDL in a subject comprising:
contacting a cell with α1PI or at least one peptide derived from α1PI; and
determining the level of LDL of said subject;
wherein the level of LDL decreases following said contact.

24. A method of designing a treatment protocol for a subject diagnosed with a disease associated with an increase in the level of LDL; comprising
determining the level of LDL in said subject diagnosed with said disease; and
comparing the level of LDL in said subject with the level of LDL of a control subject that does not have said disease;
wherein an increase in the level of LDL of said subject as compared to said control indicates that α1PI or at least one peptide derived from α1PI that is identified as capable of decreasing the level of LDL of said subject should be administered to said subject; and
wherein no increase in the level of LDL in said subject as compared to said control indicates that α1PI or at least one peptide derived from α1PI that is identified as capable of decreasing the level of LDL of said subject should not be administered to said subject.

25. The method of any one of claims 15-17, wherein said α1PI or at least one peptide derived from α1PI is administered in a therapeutically effective amount or a pharmaceutically acceptable salt or prodrug thereof, or a pharmaceutical composition comprising a therapeutically effective amount or a pharmaceutically acceptable salt or prodrug thereof, to the subject, thereby treating said disease.

26. The method of any one of claims 15-17, further comprising obtaining α1PI or at least one peptide derived from α1PI or the pharmaceutically acceptable salt or prodrug thereof.

27. The method of any one of claims 15-17 wherein said subject is a mammal.

28. The method of claim 27, wherein said subject is a human.

29. The method of claim 25, wherein said therapeutically effective amount is in the range of 5-100 μM.

30. The method of claim 25, wherein said therapeutically effective amount of said α1PI or at least one peptide derived from α1PI is administered by topical application, intravenous drip or injection, subcutaneous, intramuscular, intraperitoneal, intracranial and spinal injection, ingestion via oral route, inhalation, trans-epithelial diffusion or an implantable, time-release drug delivery device.

31. The method of any one of claims 15, 16, 20, 22, 23 and 24, wherein the results of said determining step are reported to said subject and/or a health care professional.

32. A packaged pharmaceutical comprising α1PI or at least one peptide derived from α1PI or a pharmaceutically acceptable salt or prodrug thereof which, upon administration to a subject, decreases the level of LDL of a subject.

33. A packaged pharmaceutical comprising
(a) α1PI or at least one peptide derived from α1PI or a pharmaceutically acceptable salt or prodrug thereof; and
(b) associated instructions for using said α1PI or at least one peptide derived from α1P1 to treat a disease associated with an increase in the level of LDL of a subject.

34. The packaged pharmaceutical of claim 33, wherein said α1PI or at least one peptide derived from α1PI is present as a pharmaceutical composition comprising a therapeutically effective amount or a pharmaceutically acceptable salt or prodrug thereof and a pharmaceutically acceptable carrier.

35. The packaged pharmaceutical of claim 34, further comprising a step of identifying a subject in need of said pharmaceutical.

36. The packaged pharmaceutical of claim 33, further comprising a step of identifying said α1PI or at least one peptide derived from α1PI as capable of decreasing the level of LDL in a subject.
